(19) United States
(12) Patent Application Publication
(10) Pub. No.: US 2026/0069691 A1
(43) Pub. Date: Mar. 12, 2026

(54) MODIFIED T-CELLS FOR USE IN THE TREATMENT OF HEAD AND NECK CANCER

(71) Applicant: USWM CT, LLC, Louisville, KY (US)

(72) Inventors: Stephen DAWE, Abingdon, Oxfordshire (GB); Alejandro GARCIA-CONSUEGRA LÓPEZ-PICAZO, Abingdon, Oxfordshire (GB); José María SARO SUAREZ, Abingdon, Oxfordshire (GB)

(21) Appl. No.: 19/108,854

(22) PCT Filed: Sep. 7, 2023
(86) PCT No.: PCT/GB2023/052306

§ 371 Date: Mar. 5, 2025

(30) Foreign Application Priority Data
Sep. 7, 2022 (GB) 2213051.2

(51) Int. Cl. A61K 40/11; A61K 9/00; A61K 35/17; A61K 38/00; A61K 40/32; A61K 40/42; A61K 45/06; A61P 35/04; C07K 16/28; C12N 5/0783

(52) U.S. Cl. CPC A61K 40/11; A61K 9/0019; A61K 35/17; A61K 38/00; A61K 40/32; A61K 40/4268; A61K 45/06; A61P 35/04; C07K 16/2803; C12N 5/0636; A61K 2239/31; A61K 2239/38; A61K 2239/46

(57) ABSTRACT

The disclosure relates to a method of treating head and neck cancer, and to a population of modified immunoresponsive cells expressing a heterologous TCR for use in such method.

### FIELD OF THE DISCLOSURE

The disclosure relates to a method of treating head and neck cancer, and to a population of modified immunoresponsive cells expressing a heterologous TCR for use in such method.

### BACKGROUND

Head and neck cancer is a group of cancers affecting the mouth, nose, throat, larynx, sinuses, or salivary glands and are in the majority squamous cell cancers. Together, these cancers are the seventh most-frequent cancer and the ninth most-frequent cause of death from cancer, and they globally affect more than 5.5 million people. The cancer is strongly linked to tobacco and alcohol use although other known risk factors are viral in origin and include Epstein-Barr virus or human papillomavirus infections. The mutational profile of HPV+ and HPV− head and neck cancer demonstrates that these are fundamentally distinct cancers.

Head and neck cancer commonly affects individuals of between 55 and 65 years old, males are affected twice as often as females. The average 5-year survival following diagnosis is 42-64%. Early-stage oral cancers have improved cure rates, however the majority of patients present with more advanced cancer which is less easily treated. A significant percentage of patients following first line success proceed to develop second primary tumours at a rate of 9% to 23% at 20 years, often due to the same carcinogenic exposure responsible for the original tumour.

Diagnosis is staged according to the TNM classification system, where T is the size and configuration of the tumour, N is the presence or absence of lymph node metastases, and M is the presence or absence of distant metastases. The T, N, and M characteristics are combined to produce a “stage” of the cancer, from I to IVB.

Surgical resection and radiation therapy (including 3D conformal radiation therapy, intensity-modulated radiation therapy, particle beam therapy and brachytherapy) or concomitant chemotherapy regimens are the main course of treatment for most head and neck cancers as the standard of care for tumour with regional metastases (stage III or IV). Surgery alone may suffice for early primary cancers without regional metastases (stage I or II). Typical chemotherapy agents include combinations of paclitaxel and carboplatin. Docetaxel is also an approved treatment for advanced head and neck cancer, either alone or in combination with cisplatin and/or fluorouracil.

Immune checkpoint blockade offers further options for therapy. Pembrolizumab is approved for first-line treatment of metastatic or unresectable recurrent HNSCC, and nivolumab is approved for the treatment of recurrent or metastatic HNSCC with disease progression on or after platinum-based chemotherapy. Some targeted antibody therapy options for squamous cell cancers of the head and neck include cetuximab, bevacizumab and erlotinib, including combination of cetuximab with conventional chemotherapy cisplatin. Cetuximab and platin/5-fluorouracil (5-FU) is a recognised first-line regimen.

For head and neck cancers which are recurrent, unresectable or metastatic (with no option for surgical resection or radiotherapy), preferred first-line regimens include Pembrolizumab, for instance in combination with a platinum-based chemotherapy agent (e.g. cisplatin or carboplatin) and 5-FU. Administration of Pembrolizumab as a single agent has been described for such expressing PD-L1 with a combined positivity score of ≥1.

New therapies for treating, preventing and/or delaying the progression of head and neck cancers are desired.

### SUMMARY OF THE DISCLOSURE

The present inventors have identified that use of modified T cells comprising a heterologous CD8 co-receptor and a heterologous T cell receptor capable of binding to a peptide antigen of MAGE-A4 is advantageous as a treatment for head and neck cancer. Modified T cells comprising a heterologous CD8 co-receptor and a heterologous T cell receptor capable of binding to a peptide antigen of MAGE-A4 may, for example, be comprised in a first-line treatment for head and neck cancer or relapsed head and neck cancer. The present inventors have devised exemplary treatment regimens in these respects.

Inclusion of T cell therapy in first-line treatment has the potential to modify the tumour microenvironment by infiltrating the tumour, and to further exploit the broader immune response to enhance and maintain T cell activation. This may lead to an improved frequency, depth and durability of response. Furthermore, T cell therapy in earlier lines of treatment has the advantage of reaching healthier patients, with a more favourable tumour microenvironment and with better T cells to harvest. Moreover, the healthier patient may have a greater likelihood of responding to treatment and with fewer undesired effects, such as cytokine release syndrome (CRS) and cytopenia.

The T cell therapy may be comprised in a combination therapy, such as a combination therapy that comprises a checkpoint inhibitor and/or an additional anti-cancer therapy such as a chemotherapy. Inclusion of modified T cells in combination therapy may be advantageous, as it may allow the dose of the checkpoint inhibitor or the additional anti-cancer therapy to be reduced. In turn, CRS and/or cytopenia may be reduced, especially when the anti-cancer therapy is a chemotherapy.

Accordingly, the disclosure provides a method of treating head and neck cancer in an individual, comprising administering to the individual a population of modified T cells comprising a heterologous CD8 co-receptor and a heterologous T-cell receptor (TCR) capable of binding to a peptide antigen of MAGE-A4.

The disclosure also provides a population of modified T cells comprising a heterologous CD8 co-receptor and a heterologous T-cell receptor capable of binding to a peptide antigen of MAGE-A4 for use in the method of the disclosure.

### BRIEF DESCRIPTION OF THE FIGURES

FIG. 1: Exemplary treatment regimen for locally advanced head and neck cancer that has relapsed following curative intent treatment.

FIG. 2: Alternative representation of the exemplary treatment regimen for locally advanced head and neck cancer that has relapsed following curative intent treatment.

FIG. 3: Exemplary treatment regimen for newly metastatic or unresectable locally advanced head and neck cancer.

FIG. 4: Alternative representation of the exemplary treatment regimen for newly metastatic or unresectable locally advanced head and neck cancer.

FIG. 5: Efficacy of ADP-A2M4CD8 in patients with head and neck cancer. FIG. 5A: Change in baseline sum of longest diameters of target lesion (SLD) in individual patients. FIG. 5B: Change in baseline target SLD in weeks from infusion of T-cells. The patient with stable disease had a >30% decrease In SLD, but at only one timepoint and thus was assessed as stable disease per RECIST v1.1. Data show change from baseline in SLD through progression or prior to surgical resection. Investigator-assessed best overall response indicated per RECIST v1.1. PR: partial response. RECIST: Response Evaluation Criteria in Solid Tumors. SD: stable disease.

FIG. 6: A computerised tomography scan of the hilar mass of a patient with stage IV head and neck cancer with confirmed partial response. Scans taken at baseline and at 4 weeks post-transfusion of ADP-A2M4CD8 T cells.

### DETAILED DESCRIPTION

It is to be understood that different applications of the disclosed methods and products may be tailored to the specific needs in the art. It is also to be understood that the terminology used herein is for the purpose of describing particular embodiments of the disclosure only, and is not intended to be limiting.

All publications, patents and patent applications cited herein, whether supra or infra, are hereby incorporated by reference in their entirety.

### General Definitions

Unless defined otherwise, technical and scientific terms used herein have the same meaning as commonly understood by a person skilled in the art to which this disclosure belongs.

As used in this specification and the appended claims, the singular forms “a”, “an”, and “the” include plural referents unless the content clearly dictates otherwise. Thus, for example, reference to “a TCR” includes “TCRs”, reference to “an antibody” includes two or more such antibodies, and the like.

In general, the term “comprising” is intended to mean including but not limited to. For example, the phrase “a method comprising administering a population of modified T cells” should be interpreted to mean that the method contains a step administering such a population, but that the method may contain additional such as, for example, administering a further therapeutic agent.

In some aspects of the disclosure, the word “comprising” is replaced with the phrase “consisting of”. The term “consisting of” is intended to be limiting. For example, the phrase “a method consisting of administering a population of modified T cells” should be interpreted to mean that the method contains a step administering such a population, and no additional steps.

The terms “protein” and “polypeptide” are used interchangeably herein, and are intended to refer to a polymeric chain of amino acids of any length.

Typically, the term “about” is used to refer to a value within +/−10% (such as within +/−5% or within +/−2%) of the value that follows.

For the purpose of this disclosure, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotide residues at nucleotide positions are then compared. When a position in the first sequence is occupied by the same nucleotide residue as the corresponding position in the second sequence, then the nucleotides are identical at that position. The percent identity between the two sequences is a function of the number of identical positions shared by the sequences (i.e., % identity=number of identical positions/total number of positions in the reference sequence×100).

Typically, the sequence comparison is carried out over the length of the reference sequence. For example, if the user wished to determine whether a given (“test”) sequence has a certain percentage identity to SEQ ID NO: X, SEQ ID NO: X would be the reference sequence. For example, to assess whether a sequence is at least 80% identical to SEQ ID NO: X (an example of a reference sequence), the skilled person would carry out an alignment over the length of SEQ ID NO: X, and identify how many positions in the test sequence were identical to those of SEQ ID NO: X. If at least 80% of the positions are identical, the test sequence is at least 80% identical to SEQ ID NO: X. If the sequence is shorter than SEQ ID NO: X, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm.

### Method of Treating Cancer

The disclosure provides a method of treating head and neck cancer in an individual, comprising administering to the individual a population of modified T cells comprising a heterologous CD8 co-receptor and a heterologous T-cell receptor (TCR) capable of binding to a peptide antigen of MAGE-A4. As set out above, the present inventors have identified that such a method is advantageous. In particular, modified T cells comprising a heterologous CD8 co-receptor and a heterologous T cell receptor capable of binding to a peptide antigen of MAGE-A4 may be comprised in a first-line treatment for head and neck cancer.

In the context of the disclosure, treating head and neck cancer may also encompass preventing and/or delaying the progression of head and neck cancer.

### Head and Neck Cancer in an Individual

The method of the disclosure is for treating head and neck cancer in an individual. The individual is preferably human. The individual may, for example, be a non-human mammal, such as a mouse, rat, rabbit, cat, dog, pig, cow or horse.

The head and neck cancer may be a cancer that expresses MAGE-A4. MAGE-A4 expression has been reported in head and neck cancer. For example, approximately 20-25% of solid tumours in head and neck cancer express MAGE-A4, of which 40-45% of individuals also express HLA-A*02. The cancer may, for example, be a solid tumour. The cancer may, for example, be head and neck squamous cell carcinoma.

At least 1% of the head and neck cancer cells from the individual may express MAGE-A4, such as at least 5%, at least 10%, at least 20%, at least 30%, at least 40%, at least 50%, at least 60%, at least 70% or at least 80%. The percentage of cells that express MAGE-A4 may be determined by any means known to the skilled person, such as immunohistochemistry (IHC), flow-cytometry or enzyme-linked immunosorbent assay (ELISA).

The expression of MAGE-A4 in the head and neck cancer may have an intensity of greater than or equal to (≥) 1+, such as ≥2+ or ≥3+. The intensity score may be assessed by IHC staining of the tumour, with the scoring as follows: negative=no staining or staining in less than or equal to ≤10% of the cells stained; 1+=incomplete staining in ≥10% of cells stained; 2+=weak to moderate staining in ≥10% of cells stained; strong and complete staining in ≥10% of cells stained.

The head and neck cancer may be a head and neck tumour. The head and neck cancer may be a head and neck carcinoma. The head and neck cancer may, for example, be selected from any one of head and neck squamous cell carcinoma (HNSCC), cancer of the oral cavity, cancer of the oropharynx, cancer of the hypopharynx, cancer of the throat, cancer of the larynx, cancer of the the tonsil, cancer of the tongue, cancer of the soft palate, cancer of the pharynx.

The head and neck cancer may, for example, be a nasopharyngeal cancer or a non-nasopharyngeal cancer. Non-nasopharyngeal cancers may include lip, oral cavity, oropharynx, hypopharynx, glottic larynx, supraglottic larynx, ethmoid sinus, maxillary sinus and occult primary cancers.

Accordingly, the cancer maybe nasal cancer or carcinoma or squamous cell carcinoma, including paranasal sinus and nasal cavity cancer that affects the nasal cavity and the paranasal sinuses or nasopharynx cancer including cancer which arises in the nasopharynx, the nasal cavities and the Eustachian tubes and upper part of the throat, the cancer may also be lymphoepithelioma.

The cancer may be oral cancer or carcinoma or cancer or carcinoma of the mouth including squamous cell cancer of the inner lip, lip, tongue, floor of mouth, gums, hard palate. The cancer may be throat cancer for example oropharyngeal cancer, oropharyngeal squamous cell carcinoma, HPV-positive oropharyngeal cancer or HPV-positive oropharyngeal squamous cell carcinoma, optionally wherein the squamous cell carcinoma is of the oropharynx or throat including the soft palate, the base of the tongue, and the tonsils. The cancer may be Hypopharyngeal cancer including cancer of the pyriform sinuses, the posterior pharyngeal wall, or the postericoid area or metastases thereof to the lymphatic network around the larynx. The cancer may be laryngeal cancer, including cancer of the larynx, glottic cancer, supraglottic or subglottic cancer. The cancer may be cancer of the trachea, cancer or squamous cell carcinomas of the salivary glands, teratoma, adenocarcinoma, adenoid cystic carcinoma, and mucoepidermoid carcinoma or melanomas or lymphomas of the upper aerodigestive tract. The cancer may be metastatic head and neck cancer that has metastasised to the adrenal gland, skin, liver pleura, bone, lung, or mediastinal lymph nodes.

In any case, the head and neck cancer may be primary, secondary, recurrent or relapsed, metastatic or advanced. The head and neck cancer may, for example, be recurrent or relapsed, unresectable, or metastatic head and neck cancer. For example, the head and neck cancer may not be suitable for treatment by surgical resection or radiotherapy. The head and neck cancer may, for instance, be staged as T4b,N0-3 head and neck cancer.

The head and neck cancer may, for instance, be relapsed head and neck cancer. The relapsed head and neck cancer may be a locally advanced relapse or a metastatic relapse. The head and neck cancer may, for instance, have relapsed following curative intent treatment for a locally advanced head and neck cancer. Accordingly, the individual may be a cancer patient that has received curative intent treatment of locally advanced head and neck cancer. Such cancer patients form a subpopulation of head and neck cancer patients that is well-recognised in the art. Curative intent treatment is described in detail below. The method of the disclosure may represent the first-line treatment for the relapsed head and neck cancer.

Alternatively, the head and neck cancer may, for instance, be head and neck cancer that has not previously been treated. That is, the head and neck cancer may be the first occurrence of the head and neck cancer in the individual. Accordingly, the individual may not have been treated for the head and neck cancer prior to the method of the disclosure. The head and neck cancer may, for example, be newly metastatic head and neck cancer or unresectable locally advanced head and neck cancer. The head and neck cancer may, for example, be newly metastatic head and neck cancer or unresectable locally advanced head and neck cancer that has not been previously treated. Accordingly, the individual may not have received treatment for the newly metastatic head and neck cancer or unresectable locally advanced head and neck cancer prior to the method of the disclosure. In any case, the method of the disclosure may aim to treat the previously-untreated head and neck cancer. The method of the disclosure may represent the first-line treatment for the head and neck cancer.

In the context of the disclosure, a “line” of therapy may refer to a treatment regimen for a cancer for which curative intent treatment has failed, or for which curative intent treatment may be inappropriate. Curative intent treatment is described in detail herein. Failure of a curative intent treatment may, for example, refer to failure of the curative intent treatment to eliminate the cancer or to induce remission. For instance, failure of the curative intent treatment may result in relapse, or in spread of the cancer such as local invasion or metastasis. A curative intent treatment may be inappropriate when the cancer is advanced, for instance when the cancer is a locally advanced cancer or a metastatic cancer. A “line” of therapy may therefore refer to a treatment regimen for a locally advanced, metastatic, or relapsed cancer. A “line” of therapy may refer to a treatment regimen for a cancer that is recurrent or have spread.

A “line” of therapy may, for example, be a first-line therapy for the cancer. In other words, the treatment regimen may be the first treatment regimen employed against the cancer after failure or curative intent treatment. For instance, the treatment regimen may be the first treatment regimen employed after recurrence or spread of the cancer. For example, the treatment regimen may be the first treatment regimen employed against the cancer after local advance/invasion, metastasis, or relapse. A “line” of therapy may, for example, be a second-line therapy for the cancer. In other words, the treatment regimen may be the second treatment regimen employed against the cancer, for instance after failure of the first treatment regimen. Failure of the first treatment regimen may, for example, result in recurrence or further spread of the cancer. Failure of the first treatment regimen may, for example, result in local advance/invasion, metastasis, or relapse. A line of therapy may, for example, be a third-line therapy for the cancer. In other words, the treatment regimen may be the third treatment regimen employed against the cancer, for instance after failure of the second treatment regimen. Failure of the second treatment regimen may, for example, result in recurrence or further spread of the cancer. Failure of the second treatment regimen may, for example, result in local advance/invasion, metastasis, or relapse.

### Curative Intent Treatment

Curative intent treatment may refer to any therapy that has the potential to cure the cancer in the individual. Curative intent treatment may, for example, refer to a therapy that is administered to the individual to try to cure the head and neck cancer. Curative intent treatment may precede a “line” of therapy for a cancer that has is recurrent or has spread. The cancer may therefore have recurred or spread following curative intent treatment. The cancer may have invaded locally, metastasised or relapsed following curative intent treatment.

The curative intent treatment may be any known or unknown treatment for head and neck cancer. As explained above, known treatments for head and neck cancer include surgical resection, radiation therapy and systemic therapy (e.g. chemotherapy). The curative intent treatment may therefore comprise (i) surgical resection, (ii) radiation therapy, and/or (iii) systemic therapy. For example, the curative intent treatment may comprise (i); (ii); (iii); (i) and (ii); (i) and (iii); (ii) and (iii); or (i), (ii) and (iii). In particular aspects of the disclosure, the curative intent treatment may comprise (i); (ii); or (i) and (ii).

The systemic therapy may comprise or consist of (a) a chemotherapy. The systemic therapy may comprise or consist of (b) an immunotherapy. The systemic therapy may comprise or consist of (c) a targeted therapy. For example, the systemic therapy may comprise or consist of (a); (b); (c); (a) and (b); (a) and (c); (b) and (c); or (a), (b) and (c). Chemotherapies, such as those for head and neck cancer, are well-known in the art.

Such chemotherapies may, for example, comprise a platinum-based anti-neoplastic drug, such as cisplatin or carboplatin. Such chemotherapies may, for example, comprise an anti-metabolite, such as fluorouracil (5-FU), capecitabine or methotrexate. Such chemotherapies may, for example, comprise a taxane drug, such as docetaxel or paclitaxel. Such chemotherapies may, for example, comprise a podophyllotoxin derivative, such as etoposide. Such chemotherapies may, for example, comprise an alkylating agent, such as cyclophosphamide. Such chemotherapies may, for example, comprise an anthracycline, such as doxorubicin. Such chemotherapies may, for example, comprise a vinca alkaloid, such as vincristine.

Immunotherapies, such as those for head and neck cancer, are well-known in the art. Such immunotherapies may, for example, include therapeutic immune cells, immunomodulators, checkpoint inhibitors, and vaccines. Therapeutic immune cells may include T cells, for instance engineered T cells such as CAR T cells or T cells expressing an engineered TCR. Immunomodulators may include, for example, interleukins, cytokines, chemokines, and immunomodulatory imide drugs. Immunomodulators may, for example, include Multikine (Leukocyte Interleukin, Injection). Immunomodulators, may, for example, include monalizumab, a humanized anti-NKG2A blocking antibody that prevents the inhibition of CD8+ T cells and NK cell by tumour cells expressing HLA-E. Checkpoint inhibitors may, for instance, include CTLA-4 inhibitors or PD-1 axis binding antagonists. PD-1 axis binding antagonists may, for instance, include pembrolizumab, nivolumab and penpulimab. Checkpoint inhibitors and PD-1 axis binding antagonists are described in detail below. Vaccines may include, for example, vidutolimod (CMP-001) which is a toll-like receptor 9 (TLR9) agonist cancer vaccine.

Targeted therapies, such as those for head and neck cancer, are well-known in the art. The term targeted therapy is a term of art that refers to treatments that target specific genes and proteins that help cancer cells survive and grow. Targeted therapies may, for example, include an EGFR antagonist (such as cetuximab), a tropomysin kinase receptor antagonist (such as Larotrectinib), a HER2 antagonist, a kinase inhibitor (such as afatinib or lenvatinib), a farnesyltransferase inhibitor (such as tipifarnib), a PI3 kinase inhibitor (such as buparlisib), an inhibitor of apoptosis proteins (such as xevinapant (Debio 1143)).

The systemic therapy may comprise a drug, or a combination of drugs, previously described for first-line therapy of head and neck cancer, such as head and neck cancer that is recurrent, unresectable or metastatic. The systemic therapy may comprise a drug, or a combination of drugs, previously described as a “standard-of-care” or approved treatment, such as a “standard of care” first-line treatment, for head and neck cancer, such as head and neck cancer that is recurrent, unresectable or metastatic. For example, the systemic therapy may comprise pembrolizumab, cetuximab, cisplatin, carboplatin, 5-FU, docetaxel, paclitaxel, etoposide, cyclophosphamide, doxorubicin, vincristine, methotrexate, capecitabine, and/or afatinib. The systemic therapy may, for example, comprise monotherapy with pembrolizumab, cetuximab, cisplatin, carboplatin, 5-FU, docetaxel, paclitaxel, methotrexate, or capecitabine. The systemic therapy may, for example, comprise monotherapy with: pembrolizumab and cisplatin and 5-FU; pembrolizumab and carboplatin and 5-FU; cetuximab and cisplatin and 5-FU; cetuximab and carboplatin and 5-FU; cetuximab and cisplatin; cisplatin and docetaxel; cisplatin and paclitaxel; carboplatin and docetaxel; carboplatin and paclitaxel; cisplatin and 5-FU; cisplatin and docetaxel and cetuximab; cisplatin and paclitaxel and cetuximab; carboplatin and docetaxel and cetuximab; carboplatin and paclitaxel and cetuximab; cisplatin and docetaxel and pembrolizumab; cisplatin and paclitaxel and pembrolizumab; carboplatin and docetaxel and pembrolizumab; carboplatin and paclitaxel and pembrolizumab; cetuximab and pembrolizumab; cisplatin and etoposide; carboplatin and etoposide; or cyclophosphamide and doxorubicin and vincristine.

In one aspect of the disclosure, the curative intent treatment does not comprise therapeutic T cells. Accordingly, the individual may not have received therapeutic T cells prior to the method of the disclosure.

In any case, a cancer patient that has received curative intent treatment of locally advanced head and neck cancer may have been essentially cured of the cancer by the curative intent treatment. However, head and neck cancer (such as locally advanced head and neck cancer) may be prone to relapse. For instance, the cancer may be prone to recurrence or spread. The cancer may be prone to local invasion or metastasis. The method of the disclosure may aim to treat the relapse, recurrence, spread, local invasion or metastasis. The method of the disclosure may represent the first-line treatment for the relapse, recurrence, spread, local invasion or metastasis.

### Population of Modified T Cells

The method comprises administering to the individual a population of modified T cells comprising a heterologous CD8 co-receptor and a heterologous TCR capable of binding to a peptide antigen of MAGE-A4. It is the presence of the heterologous CD8 co-receptor and a heterologous TCR that renders the T cells “modified”. The heterologous CD8 co-receptor and the heterologous TCR are typically present on the surface of the modified T cells. In other words, the modified T cells may express the heterologous CD8 co-receptor and the heterologous TCR on their surface.

In the context of the present disclosure, the term “heterologous” refers to a polypeptide or nucleic acid that is foreign to a particular biological system (such as a T cell), i.e. that is not naturally present in that system. A “heterologous” polypeptide or nucleic acid may be introduced to the system by artificial or recombinant means. Accordingly, heterologous expression of a TCR may alter the specificity of a T cell. Heterologous expression of a CD8 co-receptor may endow the T cell with functions associated with the CD8 co-receptor. The heterologous CD8 co-receptor and the heterologous TCR are described in detail below.

The modified T cells may comprise CD4+ T cells. That is, the modified T cells may comprise T cells expressing an endogenous CD4 co-receptor. The modified T cells may comprise CD8+ T cells. That is, the modified T cells may comprise T cells expressing an endogenous CD8 co-receptor. The modified T cells may comprise CD4+ T cells and CD8+ T cells. That is, the modified T cells may comprise T cells expressing an endogenous CD4 co-receptor, and comprise T cells expressing an endogenous CD8 co-receptor. Both CD4+ T cells and CD8+ T cells are capable of harbouring a heterologous CD8 co-receptor.

The modified T cells may be allogeneic with respect to the individual. The modified T cells may preferably be autologous with respect to the individual. In this case, the modified T cells may be produced by modifying endogenous cells obtained from the individual. Thus, the method may comprise producing the population. Methods for producing modified T cells are known in the art and considered in the Example below. Typically, the modified T cells of the disclosure are produced from cells, such as peripheral blood mononuclear cells (PBMCs). T cells are typically selected from the harvested cells, and manipulated to comprise the desired modifications (here, the heterologous CD8 co-receptor and the heterologous TCR). The method may therefore comprise producing the population by: (a) obtaining peripheral blood mononuclear cells (PBMCs) from the individual; (b) selecting T cells from the PBMCs; and (c) modifying the selected T cells to express the heterologous CD8 co-receptor and the heterologous TCR.

Autologous modified T cells may be produced in anticipation of the individual's need for them. That is, autologous modified T cells may be produced ahead of time, before an individual requires treatment with the modified cells. This can help to ensure that autologous modified T cells are available for administration as soon as possible after the individual is identified as requiring treatment. In this way, the individual need not wait for autologous T cells to be produced before treatment can begin. This may improve the outcome of treatment.

Production of autologous T cells ahead of time may be particularly relevant in the treatment of head and neck cancers with a high risk of relapse. The risk of relapse in an individual subjected to curative intent treatment for head and neck cancer, such as locally advanced head and neck cancer, may be determined by methods routine in the art. Such methods may include monitoring for clinical signs or symptoms. Such methods may include, for example, one or more magnetic resonance imaging (MRI), positron emission tomography (PET) and/or computerised tomography (CT) scans conducted following curative intent treatment, to monitor for progression and/or return of tumours. MRI, PET and/or CT scans may, for example, be performed about every three months (such as once every 4 to 16 weeks, or once every 8 to 12 weeks). If an individual is identified as high risk of relapse, PBMCs may be obtained at this point (i.e. prior to relapse) with a view to producing autologous modified T cells ready for administration when relapse occurs. Accordingly, when the cancer is relapsed head and neck cancer, the method of the disclosure may comprise producing the population by (a) obtaining peripheral blood mononuclear cells (PBMCs) from the individual; (b) selecting T cells from the PBMCs; and (c) modifying the selected T cells to express the heterologous TCR and optionally the heterologous CD8 co-receptor, wherein one or more of steps (a) to (c) are performed prior to relapse. Preferably, step (a) is performed prior to relapse. More preferably, steps (a) and (b) are performed prior to relapse. Most preferably, steps (a), (b) and (c) are performed prior to relapse. Any of these options effectively gives treatment a head-start on relapse.

In any case, the population of modified T cells may, for example, be administered as a single dose. The population of modified T cells may, for example, be administered as soon as possible after diagnosis of the head and neck cancer, for instance as a single dose. For example, the population of modified T cells may be administered as soon as possible after relapse of head and neck cancer is identified. The population of modified T cells may, for example, be administered as soon as possible after previously-untreated head and neck cancer is identified.

In the context of the present disclosure, the term “as soon as possible” may refer to the earliest point that it is practical to administer the population of modified T cells. As explained above, production of autologous modified T cells may take some time, potentially leading to a lag between diagnosis of the head and neck cancer and the implementation of therapy. Therefore, administration as soon as possible after diagnosis may refer to administration as soon as is practical once autologous modified T cells have been produced. Administration as soon as possible after diagnosis may, for example, refer to administration from less than about 150 days after diagnosis of the head and neck cancer, such as less than about 125 days, less than about 100 days, less than about 90 days, less than about 80 days, less than about 70 days, less than about 60 days, less than about 50 days, less than about 40 days, or less than about 30 days after diagnosis of the head and neck cancer. The population may be administered to the individual, for example, about 30 to about 150 days after diagnosis of the head and neck cancer, such as about 40 to about 125, about 50 to about 100, about 90, about 85, about 80, about 75, about 70, about 65 or about 60 days after diagnosis of the head and neck cancer.

### Heterologous TCR

The modified T cells comprise a heterologous TCR capable of binding to a peptide antigen of MAGE-A4. In other words, the modified T cells express or present a heterologous TCR capable of binding to a peptide antigen of MAGE-A4, for instance on their surface. MAGE-A4 is a well-known cancer antigen that has restricted expression in normal (i.e. non-cancerous) tissue. MAGE-A4 has been shown to repress p53 targets (such as BAX and CDKN1A) and is a binding partner for the oncogene gankyrin.

The heterologous TCR is capable of binding to a peptide antigen of MAGE-A4. The heterologous TCR may, for example, bind to GVYDGREHTV (SEQ ID NO: 1) which is a peptide sequence known as MAGE-A4 230-239 that is comprised in MAGE-A4. The heterologous TCR may, for example, bind to a complex comprising a peptide antigen of MAGE-A4 (e.g. GVYDGREHTV (SEQ ID NO: 1)) and an HLA molecule, such as an HLA-A molecule (e.g. an HLA-A*02 or an HLA-A*0201 molecule). In any case, the binding may be specific. Specificity refers to the strength of binding between the heterologous TCR and its target antigen. Specificity may be described by a dissociation constant, Kd, the ratio between bound and unbound states for the receptor-ligand system. Typically, the fewer different antigens the heterologous TCR is capable of binding other than MAGE-A4, the greater its binding specificity.

The heterologous TCR may, for example, bind to a peptide antigen of MAGE-A4 (e.g. SEQ ID NO: 1), or to a complex comprising a peptide antigen of MAGE-A4 (e.g. SEQ ID NO: 1) and an HLA molecule, with a dissociation constant (Kd) of between 0.01 μM and 100 μM, between 0.01 μM and 50 μM, between 0.01 μM and 20 μM, between 10 μM and 1000 μM, between 10 μM and 500 μM, or between 50 μM and 500 μM. For instance, in a preferred aspect of the disclosure, the heterologous TCR binds to a peptide antigen of MAGE-A4, or to a complex comprising a peptide antigen of MAGE-A4 and an HLA molecule, with a Kd of between 0.05 μM to 20.0 μM. For example, the heterologous TCR may bind to a peptide antigen of MAGE-A4, or to a complex comprising a peptide antigen of MAGE-A4 and an HLA molecule, with a Kd of 0.01 μM, 0.02 μM, 0.03 μM, 0.04 μM, 0.05 μM, 0.06 μM, 0.07 μM, 0.08 μM, 0.09 μM, 0.1 μM, 0.15M, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, 1.0 μM, 1.5 μM, 2.0 μM, 2.5 μM, 3.0 μM, 3.5 μM, 4.0 μM, 4.5 μM, 5.0 μM, 5.5 μM, 6.0 μM, 6.5 μM, 7.0 μM, 7.5 μM, 8.0 μM, 8.5 μM, 9.0 μM, 9.5 μM, 10.0 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM. The Kd may, for example, be measured using surface plasmon resonance, optionally at 25° C., optionally between a pH of 6.5 and 6.9 or 7.0 and 7.5. The dissociation constant, Kd or koff/kon may be determined by experimentally measuring the dissociation rate constant, koff, and the association rate constant, kon. A TCR dissociation constant may be measured using a soluble form of the TCR, wherein the TCR comprises a TCR alpha chain variable domain and a TCR beta chain variable domain.

The heterologous TCR may, for example, be a recombinant or synthetic or artificial TCR. That is, the heterologous TCR may be a TCR that does not exist in nature. The heterologous TCR may, for example, be an affinity enhanced TCR, for example a specific peptide enhanced affinity receptor (SPEAR™) TCR.

The heterologous TCR may, for example, comprise an alpha chain variable domain having at least 80% sequence identity to the sequence of amino acid residues 22-125 of SEQ ID NO: 2. The heterologous TCR may, for example, comprise a beta chain variable domain having at least 80% sequence identity to the sequence of amino acid residues 22-123 of SEQ ID NO: 3. The heterologous TCR may, for example, comprise an alpha chain variable domain having at least 80% sequence identity to the sequence of amino acid residues 22-125 of SEQ ID NO: 2 and a beta chain variable domain having at least 80% sequence identity to the sequence of amino acid residues 22-123 of SEQ ID NO: 3. The alpha chain variable domain may, for example, have at least 85%, at least 90%, at least 95%, at least 97%, at least 98% or at least 99% sequence identity to the sequence of amino acid residues 22-125 of SEQ ID NO: 2. The alpha chain variable domain may, for example, comprise or consist of amino acid residues 22-125 of SEQ ID NO: 2. The beta chain variable domain may, for example, have at least 85%, at least 90%, at least 95%, at least 97%, at least 98% or at least 99% sequence identity to the sequence of amino acid residues 22-123 of SEQ ID NO: 3. The beta chain amino acid sequence may, for example, comprise or consist of amino acid residues 22-123 of SEQ ID NO: 3.

The heterologous TCR may, for example, comprise an alpha chain having at least 80% sequence identity to the sequence of amino acid residues 22-282 of SEQ ID NO: 2. The heterologous TCR may, for example, comprise a beta chain having at least 80% sequence identity to the sequence of amino acid residues 22-311 of SEQ ID NO: 3. The heterologous TCR may, for example, comprise an alpha chain having at least 80% sequence identity to the sequence of amino acid residues 22-282 of SEQ ID NO: 2 and a beta chain variable domain having at least 80% sequence identity to the sequence of amino acid residues 22-311 of SEQ ID NO: 3. The alpha chain variable domain may, for example, have at least 85%, at least 90%, at least 95%, at least 97%, at least 98% or at least 99% sequence identity to the sequence of amino acid residues 22-282 of SEQ ID NO: 2. The alpha chain variable domain may, for example, comprise or consist of amino acid residues 22-282 of SEQ ID NO: 2. The beta chain variable domain may, for example, have at least 85%, at least 90%, at least 95%, at least 97%, at least 98% or at least 99% sequence identity to the sequence of amino acid residues 22-311 of SEQ ID NO: 3. The beta chain amino acid sequence may, for example, comprise or consist of amino acid residues 22-311 of SEQ ID NO: 3.

The heterologous TCR is typically expressed with N-terminal signal peptides that are cleaved prior to expression at the surface of the T cell. In this respect, amino acids 1 to 21 of each of SEQ ID NO: 2 and SEQ ID NO: 3 are typically cleaved prior to expression of the TCR at the surface of the T cell. The heterologous TCR may, for example, comprise an alpha chain amino acid sequence having at least 80% sequence identity to SEQ ID NO: 2. The heterologous TCR may, for example, comprise a beta chain amino acid sequence having at least 80% sequence identity to SEQ ID NO: 3. The heterologous TCR may, for example, comprise an alpha chain amino acid sequence having at least 80% sequence identity to SEQ ID NO: 2 and a beta chain amino acid sequence having at least 80% sequence identity to SEQ ID NO: 3. The alpha chain amino acid sequence may, for example, have at least 85%, at least 90%, at least 95%, at least 97%, at least 98% or at least 99% sequence identity to SEQ ID NO: 2. The alpha chain amino acid sequence may, for example, comprise or consist of SEQ ID NO: 2. The beta chain amino acid sequence may, for example, have at least 85%, at least 90%, at least 95%, at least 97%, at least 98% or at least 99% sequence identity to SEQ ID NO: 3. The beta chain amino acid sequence may, for example, comprise or consist of SEQ ID NO: 3.

The heterologous TCR may, for example, comprise (i) an alpha chain variable domain that comprises a CDR1 that comprises (1) the sequence of SEQ ID NO: 4 or (2) an amino acid sequence that comprises one, two or three amino acid insertions, deletions or substitutions relative to the sequence of SEQ ID NO: 4; (ii) an alpha chain variable domain that comprises a CDR2 that comprises (1) the sequence of SEQ ID NO: 5 or (2) an amino acid sequence that comprises one, two or three amino acid insertions, deletions or substitutions relative to the sequence of SEQ ID NO: 5; (iii) an alpha chain variable domain that comprises a CDR3 that comprises (1) the sequence of SEQ ID NO: 6 or (2) an amino acid sequence that comprises one, two or three amino acid insertions, deletions or substitutions relative to the sequence of SEQ ID NO: 6; (iv) a beta chain variable domain that comprises a CDR1 that comprises (1) the sequence of SEQ ID NO: 7 or (2) an amino acid sequence that comprises one, two or three amino acid insertions, deletions or substitutions relative to the sequence of SEQ ID NO:7; (v) a beta chain variable domain that comprises a CDR2 that comprises (1) the sequence of SEQ ID NO: 8 or (2) an amino acid sequence that comprises one, two or three amino acid insertions, deletions or substitutions relative to the sequence of SEQ ID NO: 8; and/or (vi) a beta chain variable domain that comprises a CDR3 that comprises (1) the sequence of SEQ ID NO: 9 or (2) an amino acid sequence that comprises one, two or three amino acid insertions, deletions or substitutions relative to the sequence of SEQ ID NO: 9.

The alpha chain of the heterologous TCR may, for example, comprise (i) an alpha chain variable domain that comprises a CDR1 that comprises (1) the sequence of SEQ ID NO: 4 or (2) an amino acid sequence that comprises one, two or three amino acid insertions, deletions or substitutions relative to the sequence of SEQ ID NO: 4; (ii) an alpha chain variable domain that comprises a CDR2 that comprises (1) the sequence of SEQ ID NO: 5 or (2) an amino acid sequence that comprises one, two or three amino acid insertions, deletions or substitutions relative to the sequence of SEQ ID NO: 5; and (iii) an alpha chain variable domain that comprises a CDR3 that comprises (1) the sequence of SEQ ID NO: 6 or (2) an amino acid sequence that comprises one, two or three amino acid insertions, deletions or substitutions relative to the sequence of SEQ ID NO: 6. The alpha chain of the heterologous TCR may, for example, comprise (i) an alpha chain variable domain that comprises a CDR1 that comprises the sequence of SEQ ID NO: 4; (ii) an alpha chain variable domain that comprises a CDR2 that comprises the sequence of SEQ ID NO: 5; and (iii) an alpha chain variable domain that comprises a CDR3 that comprises the sequence of SEQ ID NO: 6.

The beta chain of the heterologous TCR may, for example, comprise (iv) a beta chain variable domain that comprises a CDR1 that comprises (1) the sequence of SEQ ID NO: 7 or (2) an amino acid sequence that comprises one, two or three amino acid insertions, deletions or substitutions relative to the sequence of SEQ ID NO:7; (v) a beta chain variable domain that comprises a CDR2 that comprises (1) the sequence of SEQ ID NO: 8 or (2) an amino acid sequence that comprises one, two or three amino acid insertions, deletions or substitutions relative to the sequence of SEQ ID NO: 8; and (vi) a beta chain variable domain that comprises a CDR3 that comprises (1) the sequence of SEQ ID NO: 9 or (2) an amino acid sequence that comprises one, two or three amino acid insertions, deletions or substitutions relative to the sequence of SEQ ID NO: 9. The beta chain of the heterologous TCR may, for example, comprise (iv) a beta chain variable domain that comprises a CDR1 that comprises the sequence of SEQ ID NO: 7; (v) a beta chain variable domain that comprises a CDR2 that comprises the sequence of SEQ ID NO: 8; and (vi) a beta chain variable domain that comprises a CDR3 that comprises the sequence of SEQ ID NO: 9.

The heterologous TCR may, for example, comprise an alpha chain comprising a CDR1 having the sequence of SEQ ID NO: 4, a CDR2 having the sequence of SEQ ID NO: 5 and a CDR3 having the sequence of SEQ ID NO: 6, and a beta chain comprising a CDR1 having the sequence of SEQ ID NO: 7, a CDR2 having the sequence of SEQ ID NO: 8 and a CDR3 having the sequence of SEQ ID NO: 9. The heterologous TCR may, for example, have additionally any of the percentage identities in the alpha chain and beta chain discussed herein.

### Heterologous CD8 Co-Receptor

The modified T cells comprise a heterologous CD8 co-receptor. In other words, the modified T cells express a heterologous CD8 co-receptor, for instance on their surface.

CD8 is a cell surface glycoprotein that, in nature, is found on most cytotoxic T lymphocytes and mediates efficient cell-cell interactions within the immune system. CD8 acts as a co-receptor for the T cell receptor, such that CD8 and the T cell receptor together recognise antigen displayed by an antigen-presenting cell in the context of class I MHC molecules. The CD8 co-receptor binds to class 1 MHCs and potentiates TCR signaling. The functional co-receptor may be a homodimer consisting of two CD8 alpha chains, or a heterodimer consisting of one CD8 alpha chain and one CD8 beta chain.

Accordingly, the heterologous CD8 co-receptor comprised in the modified T cells may be CD8α. In other words, the heterologous CD8 co-receptor may be a homodimer consisting of two CD8 alpha chains. Alternatively, the heterologous CD8 co-receptor may be a heterodimer consisting of one CD8 alpha chain and one CD8 beta chain. In either case, a CD8 alpha chain may comprise or consist of an amino acid sequence having at least 80% (such as at least 85%, at least 90%, at least 95%, at least 97%, at least 98%, at least 99% or 100%) sequence identity to SEQ ID NO: 10. Thus, the heterologous CD8 co-receptor may comprise an amino acid sequence having at least 80% (such as at least 85%, at least 90%, at least 95%, at least 97%, at least 98%, at least 99% or 100%) sequence identity to SEQ ID NO: 10.

CD8 alpha chains and CD8 beta chains both share significant homology to immunoglobulin variable light chains. CD8 alpha chains and beta chains have CDR-like loops involved in MHC-Class I binding. The heterologous CD8 co-receptor may, for example, comprise a CD8 alpha chain that comprises: (i) an alpha chain CDR1 that comprises (1) the sequence of SEQ ID NO: 11 or (2) an amino acid sequence that comprises one, two or three amino acid insertions, deletions or substitutions relative to the sequence of SEQ ID NO: 11; (ii) an alpha chain CDR2 that comprises (1) the sequence of SEQ ID NO: 12 or (2) an amino acid sequence that comprises one, two or three amino acid insertions, deletions or substitutions relative to the sequence of SEQ ID NO: 12; and/or (iii) an alpha chain CDR3 that comprises (1) the sequence of SEQ ID NO: 13 or (2) an amino acid sequence that comprises one, two or three amino acid insertions, deletions or substitutions relative to the sequence of SEQ ID NO: 13.

The heterologous CD8 co-receptor is capable of binding to a class I MHC molecule. The heterologous CD8 co-receptor may, for example, bind to the as portion of a class I MHC molecule, for instance via the IgV-like domain of the CD8 co-receptor. The α₃ portion is typically found between residues 223 and 229 of a class I MHC molecule. The ability of the heterologous CD8 co-receptor to bind to a class I MHC molecule improves the ability of the modified T cells to engage cognate antigen via their heterologous TCR. The cognate antigen, MAGE-A4, is typically presented in complex with a class I MHC molecule such as HLA-A*02. The heterologous CD8 co-receptor may improve or increase the off-rate (k_{off}) of the TCR/peptide-MHCI interaction in the modified cells. The improvement or increase may be relative to modified T cells that comprise a heterologous TCR that binds to MAGE-A4 but which lack a heterologous CD8 co-receptor. The heterologous CD8 co-receptor may, for example, assist in organising the heterologous TCR on the surface of modified cells, thereby improving the ability of the heterologous TCR to participate in the TCR/peptide-MHCI interaction. The heterologous CD8 co-receptor may, for example, bind or interact with LCK (lymphocyte-specific protein tyrosine kinase) in a zinc-dependent manner leading to activation of transcription factors like NFAT, NF-1B, and AP-1. Accordingly, expression of a heterologous CD8 co-receptor may confer upon the modified T cells an improved affinity and/or avidity for MAGE-A4, and/or improved activation upon binding to MAGE-A4. Methods for determining affinity, avidity and T cell activation are well-known in the art. Expression of a heterologous CD8 co-receptor may confer upon the modified T cells an improved or increased expression of CD40L, cytokine production, cytotoxic activity, induction of dendritic cell maturation or induction of dendritic cell cytokine production, for instance in response to antigen (MAGE-A4) binding. Improvements or increases may be relative to modified T cells that comprise a heterologous TCR that binds to a peptide antigen of MAGE-A4 but which lack a heterologous CD8 co-receptor.

Synergy has been demonstrated between CD8a and peptide antigen presented on HLA-A*0201. Therefore, in one aspect of the disclosure, the heterologous CD8 co-receptor may be CD8a and the heterologous TCR may be capable of binding to a peptide antigen of MAGE-A4 in complex with HLA-A*0201. The peptide antigen may, for example, be SEQ ID NO: 1.

### Checkpoint Inhibitors

The method may comprise administering a checkpoint inhibitor to the individual. Therefore, the method may comprise administering to the individual (i) a population of modified T cells comprising a heterologous CD8 co-receptor and a heterologous T-cell receptor (TCR) capable of binding to a peptide antigen of MAGE-A4, and (ii) a checkpoint inhibitor. In other words, the method may comprise combination treatment with individual (i) a population of modified T cells comprising a heterologous CD8 co-receptor and a heterologous T-cell receptor (TCR) capable of binding to a peptide antigen of MAGE-A4, and (ii) a checkpoint inhibitor. An additional anti-cancer therapy, such as a chemotherapy, may also be included in the combination as set out below.

The checkpoint inhibitor may be administered to the individual in the same line of therapy as the population of modified T cells. As set out above, a “line” of therapy may refer to a particular treatment regimen for a cancer for which curative intent treatment has failed, or for which curative intent treatment may be inappropriate. Thus, the checkpoint inhibitor and the population of modified T cells may be administered as part of the same treatment regimen for a cancer for which curative intent treatment has failed, or for which curative intent treatment may be inappropriate. The checkpoint inhibitor and the population of modified T cells may be administered as part of a first-line treatment regimen. The checkpoint inhibitor and the population of modified T cells may be administered as part of a second-line treatment regimen. The second-line treatment regimen may, for example, be employed following failure of the first-line treatment regimen. The checkpoint inhibitor and the population of modified T cells may be administered as part of a third-line treatment regimen. The third-line treatment regimen may, for example, be employed following failure of the second-line treatment regimen.

Checkpoint inhibitor therapy is a form of cancer immunotherapy. The therapy targets immune checkpoints, key regulators of the immune system that when stimulated can dampen the immune response to an immunologic stimulus. Checkpoint therapy can block inhibitory checkpoints, restoring immune system function. Checkpoint inhibitors can target the molecules CTLA4, PD-1 and PD-L1.

The checkpoint inhibitor may, for example, target CTLA4. That is, the checkpoint inhibitor may comprise a CTLA4 blocker. CTLA4 blockers are known in the art and include, for example, ipilimumab.

The checkpoint inhibitor may comprise a PD-1 axis binding antagonist. Administration of PD-1 axis binding antagonists is described in the art as a standard-of-care treatment for recurrent, unresectable or metastatic head and neck cancers, e.g. non-nasopharyngeal cancers including lip, oral cavity, oropharynx, hypopharynx, glottic larynx, supraglottic larynx, ethmoid sinus, maxillary sinus and occult primary cancers. For example, pembrolizumab may be administered as the standard-of-care treatment for recurrent, unresectable or metastatic head and neck cancers having tumours that express PD-L1 with CPS ≥1. Pembrolizumab may be administered with a platinum-based agent (e.g. cisplatin or carboplatin) and 5-fluorouracil as the standard-of-care treatment for recurrent, unresectable or metastatic head and neck cancers. In some circumstances, pembrolizumab may be administered with a platinum-based agent (e.g. cisplatin or carboplatin), and docetaxel or paclitaxel as a first line or subsequent line standard-of-care treatment for recurrent, unresectable or metastatic head and neck cancers. In certain circumstances, pembrolizumab may be administered with cetuximab as a first line or subsequent line standard-of-care treatment for recurrent, unresectable or metastatic head and neck cancers. In certain circumstances, pembrolizumab may be administered as a first line or subsequent line standard-of-care treatment for recurrent, unresectable or metastatic head and neck cancers having high microsatellite instability (MSI-H), such as cancers having DNA mismatch repair (MMR). In some circumstances, pembrolizumab or nivolumab may be administered as a subsequent line standard-of-care treatment for recurrent, unresectable or metastatic head and neck cancers if the cancer has progressed during or after treatment with a platinum-based agent.

Programmed cell death protein 1 (PD-1, also known as CD279) is a protein that is expressed on the surface of T cells and has a role in regulating immune responses by maintaining T cell homeostasis. Ligation of PD-1 to one of its ligands (PD-L1 or PD-L2) transmits an inhibitory signal within the T cell. In particular, PD-1-generated signals prevent phosphorylation of key TCR signalling intermediates, thereby terminating early TCR signalling and reducing T cell activation. T cell effector functions (such as proliferation, cytotoxicity and cytokine production) are reduced, and the ability to transition to memory T cells is impaired.

PD-L1 and PD-L2 are members of the B7 family. PD-L1 protein is upregulated on certain activated immune cells (such as macrophages, dendritic cells, T cells and B cells), and is also expressed upon certain normal tissues. PD-L1 is also highly expressed in many cancers. PD-L2 is expressed mainly by dendritic cells and some tumours. As many cancers express PD-1 ligands, the PD-1 axis has an established role in cancer immune evasion and tumour resistance.

Expression of PD-1 ligands by cancers, such as solid tumours, renders the tumour microenvironment immunosuppressive. The function of modified T cells infiltrating the tumour may therefore be inhibited. Endogenous anti-tumour T cell responses may also be inhibited. In this way, tumours are more able to evade the immune system. In the present disclosure, a PD-1 axis binding antagonist may be administered to counteract suppressive effects of PD-L1 and/or PD-L2 expression in the tumour microenvironment. By counteracting suppression, the function of modified and/or endogenous T cells may be sustained. That is, administration of a PD-1 axis binding antagonist may sustain the function of modified T cells comprised in the administration, and/or their descendants. Administration of a PD-1 axis binding antagonist may sustain the function of endogenous T cells in the individual. Administration of a PD-1 axis binding antagonist may sustain the function of modified T cells comprised in the administration (and/or their descendants), and of endogenous T cells in the individual. In any case, the endogenous T cells may, for example, be comprised in the tumour microenvironment. For instance, the endogenous T cells may be tumour infiltrating lymphocytes (TILs).

Sustaining the function of T cells may refer, for example, to maintaining, restoring and/or enhancing T cell function. Sustaining T cell function may, for example, refer to sustaining T cell activation. In this way, the duration of an effective T cell response may be extended. In other words, sustained activation maybe associated with an improved duration of effector function (such as cytokine production, cytotoxicity and/or proliferation). Sustained activation may also assist the T cells' ability to transition to memory T cells. The generation of memory T cells is advantageous, as it permits anti-tumour immunity to be maintained in the long-term e.g. for months or years. Methods for determining activation, cytokine production, cytotoxicity, proliferation, and generation of memory T cells are well-known in the art.

Administration of the PD-1 axis binding antagonist may sustain the function of the modified T cells and/or endogenous T cells by reducing exhaustion. Exhaustion may be reduced within the population of modified T cells, and/or within T cells descended from the population of modified T cells. Exhaustion may be reduced within endogenous T cells in the individual. Exhaustion may be reduced (i) within the population of modified T cells and/or within T cells descended from the population of modified T cells, and (ii) within endogenous T cells in the individual. Exhausted T cells typically express high levels of PD-1, and experience a loss of function. For instance, exhausted T cells may have reduced ability to produce cytokines such as IL-2 or TNFα. Exhausted T cells may have reduced proliferative capacity. Exhausted T cells may have reduced cytotoxic potential. Ultimately, exhausted T cells may be targeted for destruction. Exhaustion therefore causes loss of T cell function, or loss of T cells themselves, which is disadvantageous to tumour immunity. Reducing T cell exhaustion may therefore improve therapeutic outcome.

In the context of the disclosure, a PD-1 axis binding antagonist is a molecule that inhibits the interaction of PD-1 with a PD-1 ligand, and/or transduction of a signal resulting from the interaction of PD-1 with a PD-1 ligand. The PD-1 ligand may be PD-L1 or PD-L2. The PD-1 axis binding antagonist may, for example, reduce or prevent the interaction of PD-1 with a PD-1 ligand. The PD-1 axis binding antagonist may, for example, reduce or prevent transduction of a signal resulting from the interaction of PD-1 with a PD-1 ligand. The PD-1 axis binding antagonist may block, inhibit or reduce the biological activity of PD-1 and/or a PD-1 ligand.

By inhibiting the interaction of PD-1 with a PD-1 ligand, and/or transduction of a signal resulting from the interaction of PD-1 with a PD-1 ligand, the PD-1 axis binding antagonist may sustain (e.g. maintain, restore or enhance) the function of endogenous T cells. In the same way, the PD-1 axis binding antagonist may sustain (e.g. maintain, restore or enhance) the function of modified T cells administered to the individual. Sustained function may, for example, be indicated by maintenance of, or improvements in, T-cell proliferation, cytokine production, target cell killing, activation, CD28 signalling, ability to infiltrate tumour, ability to recognise and bind to dendritic cell presented antigen, and/or ability to produce interferon. In this way, the PD-1 axis binding antagonist counteracts the immunosuppressive nature of the tumour microenvironment.

The PD-1 axis binding antagonist may, for example, be a PD-1 binding antagonist. That is, the PD-1 axis binding antagonist may inhibit (e.g. prevent or reduce) binding of PD-1 to its binding partners. For example, the PD-1 axis binding antagonist may inhibit the binding of PD-1 to PD-L1, PD-L2, or both PD-L1 and PD-L2. The PD-1 binding antagonist may, for example, be an antibody that binds to PD-1, or an antigen-binding variant or fragment thereof. The PD-1 binding antagonist may, for example, be an antibody that binds to SEQ ID NO: 14, or an antigen-binding variant or fragment thereof. Antibodies that bind to PD-1 are well-known in the art and include, for example, nivolumab, pembrolizumab, cemiplimab, dostarlimab, JTX-4014, spartalizumab, camrelizumab, sintilimab, tislelizumab, toripalimab, retifanlimab (INCMGA00012), AMP-224, MEDI0680 (AMP-514), sasanlimab, budigalimab, ezabenlimab (BI 754091), and zimberelimab (AB122). The PD-1 axis binding antagonist may therefore be nivolumab, pembrolizumab, cemiplimab, dostarlimab, JTX-4014, spartalizumab, camrelizumab, sintilimab, tislelizumab, toripalimab, retifanlimab (INCMGA00012), AMP-224, MEDI0680 (AMP-514), sasanlimab, budigalimab, ezabenlimab (BI 754091) or zimberelimab (AB122), or any combination thereof. The PD-1 axis binding antagonist may, for example, be pembrolizumab. The PD-1 binding antagonist may alternatively be any immunoadhesin, protein or oligopeptide that inhibits, decreases, prevent, or interferes with signal transduction due to the interaction of PD-1 with PD-L1 and/or PD-L2.

The heavy chain sequence and light chain sequence of nivolumab are set out in SEQ ID NOs: 17 and 18 respectively. The heavy chain sequence and light chain sequence of pembrolizumab are set out in SEQ ID NOs: 19 and 20 respectively. The heavy chain sequence and light chain sequence of cemiplimab are set out in SEQ ID NOs: 21 and 22 respectively. The heavy chain sequence and light chain sequence of dostarlimab are set out in SEQ ID NOs: 31 and 32 respectively. A skilled person equipped with the heavy and light chain sequences of a given antibody may identify antigen-binding variants or fragments of the antibody using methods routine in the art. An antigen-binding variant or fragment of nivolumab may, for example, comprise the three CDRs comprised in SEQ ID NO: 17 and the three CDRs comprised in SEQ ID NO: 18. An antigen-binding variant or fragment of pembrolizumab may, for example, comprise the three CDRs comprised in SEQ ID NO: 19 and the three CDRs comprised in SEQ ID NO: 20. An antigen-binding variant or fragment of cemiplimab may, for example, comprise the three CDRs comprised in SEQ ID NO: 21 and the three CDRs comprised in SEQ ID NO: 22. An antigen-binding variant or fragment of dostarlimab may, for example, comprise the three CDRs comprised in SEQ ID NO: 31 and the three CDRs comprised in SEQ ID NO: 32. Methods for identifying CDRs within a heavy chain or light chain sequence are routine in the art.

The PD-1 axis binding antagonist may, for example, be a PD-L1 binding antagonist. That is, the PD-1 axis binding antagonist may inhibit (e.g. prevent or reduce) binding of PD-L1 to a binding partner. For example, the PD-1 axis binding antagonist may inhibit the binding of PD-L1 to PD-1. The PD-L1 binding antagonist may, for example, be an antibody that binds PD-L1, or an antigen-binding variant or fragment thereof. The PD-L1 binding antagonist may, for example, be an antibody that binds to SEQ ID NO: 15, or an antigen-binding variant or fragment thereof. Antibodies that bind to PD-L1 are well-known in the art and include, for example, durvalumab, atezolizumab, avelumab, BMS 936559 (MDX-1105), envafolimab (KN035) and cosibelimab (CK-301). The PD-L1 axis binding antagonist may therefore be durvalumab, atezolizumab, avelumab, BMS 936559 (MDX-1105), envafolimab (KN035) or cosibelimab (CK-301), or any combination thereof. The PD-L1 binding antagonist may alternatively be any immunoadhesin, protein or oligopeptide that inhibits, decreases, prevent, or interferes with signal transduction due to the interaction of PD-L1 with PD-1.

The heavy chain sequence and light chain sequence of durvalumab are set out in SEQ ID NOs: 23 and 24 respectively. The heavy chain sequence and light chain sequence of atezolizumab are set out in SEQ ID NOs: 25 and 26 respectively. The heavy chain sequence and light chain sequence of avelumab are set out in SEQ ID NOs: 27 and 28 respectively. The heavy chain sequence and light chain sequence of BMS 936559 (MDX-1105) are set out in SEQ ID NOs: 29 and 30 respectively. A skilled person equipped with the heavy and light chain sequences of a given antibody may identify antigen-binding variants or fragments of the antibody using methods routine in the art. An antigen-binding variant or fragment of durvalumab may, for example, comprise the three CDRs comprised in SEQ ID NO: 23 and the three CDRs comprised in SEQ ID NO: 24. An antigen-binding variant or fragment of atezolizumab may, for example, comprise the three CDRs comprised in SEQ ID NO: 25 and the three CDRs comprised in SEQ ID NO: 26. An antigen-binding variant or fragment of avelumab may, for example, comprise the three CDRs comprised in SEQ ID NO: 27 and the three CDRs comprised in SEQ ID NO: 28. An antigen-binding variant or fragment of BMS 936559 (MDX-1105) may, for example, comprise the three CDRs comprised in SEQ ID NO: 29 and the three CDRs comprised in SEQ ID NO: 30. Methods for identifying CDRs within a heavy chain or light chain sequence are routine in the art.

The PD-1 axis binding antagonist may, for example, be a PD-L2 binding antagonist. That is, the PD-1 axis binding antagonist may inhibit (e.g. prevent or reduce) binding of PD-L2 to a binding partner. The PD-L2 binding antagonist may, for example, be an antibody that binds PD-L2, or an antigen-binding variant or fragment thereof. The PD-L2 binding antagonist may, for example, be an antibody that binds to SEQ ID NO: 16, or an antigen-binding variant or fragment thereof. The PD-L2 binding antagonist may alternatively be any immunoadhesin, protein or oligopeptide that inhibits, decreases, prevent, or interferes with signal transduction due to the interaction of PD-L2 with PD-1.

When the PD-1 axis binding antagonist is an antibody (such as a known antibody, or an antigen-binding variant thereof), the PD-1 axis binding antagonist may be a monoclonal antibody, a human or humanised antibody, a full-length antibody, a diabody, a linear antibody, or a single-chain antibody molecule, for example. The antibody isotype may be selected from any of the five classes of immunoglobulins: IgA, IgD, IgE, IgG and IgM, having heavy chains designated alpha, delta, epsilon, gamma and mu (M), respectively. The gamma and alpha class antibodies may be of any of subclasses: IgG1, IgG2A, IgG2B, IgG3, IgG4, IgA1 and IgA2. When the PD-1 axis binding antagonist is an antigen-binding fragment of an antibody, the PD-1 axis binding antagonist may be a Fv, Fab, Fab′, Fab′-SH, F(ab′)2, or scFv, for example.

When the PD-1 axis binding antagonist is an immunoadhesin, the immunoadhesin may comprise an adhesin domain conferring binding activity for a PD-1 axis component (e.g. PD-1, PD-L1, or PD-L2) and an immunoglobulin constant domain. The immunoglobulin constant domain may be from any isotype, such as IgG1, IgG2, IgG2A, IgG2B, IgG3, IgG4 subtypes, IgA, IgA1, IgA2, IgE, IgD or IgM. The immunoglobulin constant domain may, for example, comprise (i) the hinge, CH2 and CH3, or (ii) the hinge, CH1, CH2 and CH3 regions of an immunoglobulin molecule. Accordingly, the immunoadhesin may comprise (a) the extracellular or PD-1 binding portions of PD-L1 or PD-L2, or the extracellular or PD-L1 or PD-L2 binding portions of PD-1, fused to (b) a constant domain of an immunoglobulin sequence.

At least 1% of the head and neck cancer cells from the individual may express PD-L1, such as at least 5%, at least 10%, at least 20%, at least 30%, at least 40%, at least 50%, at least 60%, at least 70% or at least 80%. The head and neck cancer from the individual may express PD-L1 with a tumour proportion score (TPS) of greater than or equal to (≥) 1%, such as ≥2%, 10% or as ≥50%. The percentage of cells that express PD-L1 may be determined by any means known to the skilled person, such as IHC, flow-cytometry or ELISA.

At least 1% of the head and neck cancer cells from the individual may express PD-L2, such as at least 5%, at least 10%, at least 20%, at least 30%, at least 40%, at least 50%, at least 60%, at least 70% or at least 80%. The head and neck cancer from the individual may express PD-L2 with a tumour proportion score (TPS) of greater than or equal to (≥) 1%, such as ≥2%, 10% or as ≥50%. The percentage of cells that express PD-L2 may be determined by any means known to the skilled person, such as IHC, flow-cytometry or ELISA.

The expression of PD-1, PD-L1 and/or PD-L2 in the head and neck cancer may have an intensity of greater than or equal to (≥) 1+, such as ≥2+ or ≥3+. The intensity score may be assessed by IHC staining of the tumour, with the scoring as follows: negative=no staining or staining in less than or equal to ≤10% of the cells stained; 1+=incomplete staining in ≥10% of cells stained; 2+=weak to moderate staining in ≥10% of cells stained; strong and complete staining in ≥10% of cells stained.

The population of modified T cells and the checkpoint inhibitor may be administered any number of times, and in any order. The population of modified T cells may, for example, be administered as a single dose. The checkpoint inhibitor may, for example, be administered (a) before the modified T cells, (b) at the same time as the modified T cells, and/or (c) after the modified T cells. For instance, the checkpoint inhibitor may, for example, be administered: (a); (b); (c); (a) and (b); (a) and (c); (b) and (c); or (a), (b) and (c). When the checkpoint inhibitor is administered at the same time as the population of modified T cells, the checkpoint inhibitor and the population of modified T cells may be comprised in the same composition or in separate compositions.

Administration of the checkpoint inhibitor at the same time as the modified T cells may refer to administration of the checkpoint inhibitor and the modified T cells at substantially the same time. In other words, a dose of the checkpoint inhibitor may be administered at about the same time as a dose of the population of modified T cells. For instance, a dose of the checkpoint inhibitor may be administered within about 2 hours, about 4 hours, about 6 hours, about 8 hours, about 12 hours, or about 24 hours as a dose of the population of modified T cells.

Administration of the checkpoint inhibitor before the modified T cells may refer to administration of the checkpoint inhibitor at any time before the modified T cells. In other words, a dose of the checkpoint inhibitor may be administered at any time before a dose of the population of modified T cells. For instance, a dose of the checkpoint inhibitor may be administered about 24 hours or more, about 36 hours or more, about 48 hours or more, about 72 hours or more, about 4 days or more, about 5 days or more, about 6 days or more, about one week or more, about two weeks or more, about three weeks or more, or about four weeks or more, about five weeks or more, about six weeks or more, about 8 weeks or more, or about 12 weeks or more, before the population of modified T cells. The checkpoint inhibitor may, for example, be administered before the modified T cells in order to initiate treatment while autologous modified T cells are produced.

Administration of the checkpoint inhibitor after the modified T cells may refer to administration of the checkpoint inhibitor at any time after the modified T cells. In other words, a dose of the checkpoint inhibitor may be administered at any time after a dose of the population of modified T cells. For instance, a dose of the checkpoint inhibitor may be administered about 24 hours or more, about 36 hours or more, about 48 hours or more, about 72 hours or more, about 4 days or more, about 5 days or more, about 6 days or more, about one week or more, about two weeks or more, about three weeks or more, or about four weeks or more, about five weeks or more, about six weeks or more, about 8 weeks or more, or about 12 weeks or more, after the population of modified T cells.

If the checkpoint inhibitor is administered before the population of modified T cells, the checkpoint inhibitor may be continued after administration of the modified T cells. Thus, in one aspect of the disclosure, administration of the checkpoint inhibitor begins before administration of the population of modified T cells, and continues after administration of the population of modified T cells. In other words, a dose of the checkpoint inhibitor may be administered before a dose of the population of modified T cells, and one or more further doses of the checkpoint inhibitor may be administered later. The doses of the checkpoint inhibitor may, for example, be administered in accordance with a known treatment regime for the checkpoint inhibitor. The purpose of the further doses of the checkpoint inhibitor may be to maintain the effects achieved by administration of the initial dose. Each of the one or more further doses may comprise the same checkpoint inhibitor as the initial dose, or a different checkpoint inhibitor from the initial dose.

If the checkpoint inhibitor is administered at the same time as the population of modified T cells, the checkpoint inhibitor may be continued after administration of the modified T cells. Thus, in one aspect of the disclosure, administration of the checkpoint inhibitor begins at the same time as administration of the population of modified T cells, and continues after administration of the population of modified T cells. In other words, a dose of the checkpoint inhibitor may be administered at the same time as a dose of the population of modified T cells, and one or more further doses of the checkpoint inhibitor may be administered later. The doses of the checkpoint inhibitor may, for example, be administered in accordance with a known treatment regime for the checkpoint inhibitor. The purpose of the further doses of the checkpoint inhibitor may be to maintain the effects achieved by administration of the initial dose. Each of the one or more further doses may comprise the same checkpoint inhibitor as the initial dose, or a different checkpoint inhibitor from the initial dose.

If the checkpoint inhibitor is administered after the population of modified T cells, the checkpoint inhibitor may be continued after initial administration. Thus, in one aspect of the disclosure, administration of the checkpoint inhibitor begins after administration of the population of modified T cells, and continues after initial administration of the checkpoint inhibitor. In other words, a dose of the checkpoint inhibitor may be administered after a dose of the population of modified T cells, and one or more further doses of the checkpoint inhibitor may be administered later. The doses of the checkpoint inhibitor may, for example, be administered in accordance with a known treatment regime for the checkpoint inhibitor. The purpose of the further doses of the checkpoint inhibitor may be to maintain the effects achieved by administration of the initial dose. Each of the one or more further doses may comprise the same checkpoint inhibitor as the initial dose, or a different checkpoint inhibitor from the initial dose.

In any case, the one or more further doses of the checkpoint inhibitor may be administered at any appropriate interval. Suitable dosage intervals for checkpoint inhibitor are known in the art and may be peculiar to the identity of the checkpoint inhibitor. For example, for a PD-1 axis binding antagonist such as pembrolizumab, the one or more further doses may, for example, be administered about once every two weeks (Q2W) beginning two weeks from administration of the initial dose. The one or more further doses may, for example, be administered about once every three weeks (Q3W) beginning three weeks from administration of the initial dose. The one or more further doses may, for example, be administered about once every four weeks (Q4W) beginning four weeks from administration of the initial dose. The one or more further doses may, for example, be administered about once every five weeks (Q5W) beginning five weeks from administration of the initial dose. The one or more further doses may, for example, be administered about once every six weeks (Q6W) beginning six weeks from administration of the initial dose. In a preferred aspect of the disclosure, the one or more further doses is administered about once every three weeks (Q3W) beginning three weeks from administration of the initial dose, or about once every six weeks (Q6W) beginning six weeks from administration of the initial dose.

Any number of further doses of the checkpoint inhibitor may be administered, such as one or more, two or more, three or more, four or more, five or more, six or more, seven or more, eight or more, nine or more, ten or more, 15 or more, 20 or more, 25 or more, 30 or more, 35 or more, 40 or more, 45 or more or 50 or more further doses. Further doses may be administered until disease progression, unacceptable toxicity, withdrawal of consent, or death.

### Additional Anti-Cancer Therapy

The method may comprise administering an additional anti-cancer therapy to the individual. Therefore, the method may comprise administering to the individual (i) a population of modified T cells comprising a heterologous CD8 co-receptor and a heterologous T-cell receptor (TCR) capable of binding to a peptide antigen of MAGE-A4, and (ii) an additional anti-cancer therapy. In other words, the method may comprise combination treatment with individual (i) a population of modified T cells comprising a heterologous CD8 co-receptor and a heterologous T-cell receptor (TCR) capable of binding to a peptide antigen of MAGE-A4, and (ii) an additional anti-cancer therapy. A checkpoint inhibitor, such as a PD-1 axis binding antagonist, may also be included in the combination as set out above.

The additional anti-cancer therapy may be administered to the individual in the same line of therapy as the population of modified T cells. As set out above, a “line” of therapy may refer to a particular treatment regimen for a cancer for which curative intent treatment has failed, or for which curative intent treatment may be inappropriate. Thus, the additional anti-cancer therapy and the population of modified T cells may be administered as part of the same treatment regimen for a cancer for which curative intent treatment has failed, or for which curative intent treatment may be inappropriate. The additional anti-cancer therapy and the population of modified T cells may be administered as part of a first-line treatment regimen. The additional anti-cancer therapy and the population of modified T cells may be administered as part of a second-line treatment regimen. The second-line treatment regimen may, for example, be employed following failure of the first-line treatment regimen. The additional anti-cancer therapy and the population of modified T cells may be administered as part of a third-line treatment regimen. The third-line treatment regimen may, for example, be employed following failure of the second-line treatment regimen.

The additional anti-cancer therapy may, for example, be an anti-cancer drug therapy. In other words, the additional anti-cancer therapy may be a systemic therapy. The additional anti-cancer therapy may, for example, comprise or consist of (a) a chemotherapy. The additional anti-cancer therapy may, for example, comprise or consist of (b) an immunotherapy. The additional anti-cancer therapy may, for example, comprise or consist of (c) a targeted therapy. For example, the additional anti-cancer therapy may comprise or consist of (a); (b); (c); (a) and (b); (a) and (c); (b) and (c); or (a), (b) and (c).

Any chemotherapy may be comprised in the additional anti-cancer therapy. Chemotherapies, such as those for head and neck cancer, are well-known in the art. Such chemotherapies may, for example, comprise a platinum-based anti-neoplastic drug, such as cisplatin or carboplatin. Such chemotherapies may, for example, comprise an anti-metabolite, such as fluorouracil (5-FU), capecitabine or methotrexate. Such chemotherapies may, for example, comprise a taxane drug, such as docetaxel or paclitaxel. Such chemotherapies may, for example, comprise a podophyllotoxin derivative, such as etoposide. Such chemotherapies may, for example, comprise an alkylating agent, such as cyclophosphamide. Such chemotherapies may, for example, comprise an anthracycline, such as doxorubicin. Such chemotherapies may, for example, comprise a vinca alkaloid, such as vincristine.

Any immunotherapy may be comprised in the additional anti-cancer therapy. Immunotherapies, such as those for head and neck cancer, are well-known in the art. Such immunotherapies may, for example, include therapeutic immune cells, immunomodulators, checkpoint inhibitors, and vaccines. Therapeutic immune cells may include T cells, for instance engineered T cells such as CAR T cells or T cells expressing an engineered TCR. Immunomodulators may include, for example, interleukins, cytokines, chemokines, and immunomodulatory imide drugs. Immunomodulators may, for example, include Multikine (Leukocyte Interleukin, Injection). Immunomodulators, may, for example, include monalizumab, a humanized anti-NKG2A blocking antibody that prevents the inhibition of CD8+ T cells and NK cell by tumour cells expressing HLA-E. Checkpoint inhibitors may, for instance, include CTLA-4 inhibitors or PD-1 axis binding antagonists. PD-1 axis binding antagonists may, for instance, include pembrolizumab, nivolumab and penpulimab. Checkpoint inhibitors and PD-1 axis binding antagonists are described in detail herein. Vaccines may include, for example, vidutolimod (CMP-001) which is a toll-like receptor 9 (TLR9) agonist cancer vaccine.

Any targeted therapy may be comprised in the additional anti-cancer therapy. Targeted therapies, such as those for head and neck cancer, are well-known in the art. Targeted therapies may, for example, include an EGFR antagonist (such as cetuximab), a tropomysin kinase receptor antagonist (such as Larotrectinib), a HER2 antagonist, a kinase inhibitor (such as afatinib or lenvatinib), a farnesyltransferase inhibitor (such as tipifarnib), a PI3 kinase inhibitor (such as buparlisib), an inhibitor of apoptosis proteins (such as xevinapant (Debio 1143)).

The additional anti-cancer therapy may comprise a drug, or a combination of drugs, previously described for first-line therapy of head and neck cancer, such as head and neck cancer that is recurrent, unresectable or metastatic. The anti-cancer therapy may comprise a drug, or a combination of drugs, previously described as an approved or “standard-of-care” treatment (such as a first-line “standard of care” treatment) for head and neck cancer, such as head and neck cancer that is recurrent, unresectable or metastatic. In this context, approval may relate to approval by the FDA, EMA or MHRA for example. For example, the additional anti-cancer therapy may comprise pembrolizumab, cetuximab, cisplatin, carboplatin, 5-FU, docetaxel, paclitaxel, etoposide, cyclophosphamide, doxorubicin, vincristine, methotrexate, capecitabine, and/or afatinib. The anti-cancer therapy may, for example, comprise monotherapy with pembrolizumab, cetuximab, cisplatin, carboplatin, 5-FU, docetaxel, paclitaxel, methotrexate, or capecitabine. The anti-cancer therapy may, for example, comprise monotherapy with: pembrolizumab and cisplatin and 5-FU; pembrolizumab and carboplatin and 5-FU; cetuximab and cisplatin and 5-FU; cetuximab and carboplatin and 5-FU; cetuximab and cisplatin; cisplatin and docetaxel; cisplatin and paclitaxel; carboplatin and docetaxel; carboplatin and paclitaxel; cisplatin and 5-FU; cisplatin and docetaxel and cetuximab; cisplatin and paclitaxel and cetuximab; carboplatin and docetaxel and cetuximab; carboplatin and paclitaxel and cetuximab; cisplatin and docetaxel and pembrolizumab; cisplatin and paclitaxel and pembrolizumab; carboplatin and docetaxel and pembrolizumab; carboplatin and paclitaxel and pembrolizumab; cetuximab and pembrolizumab; cisplatin and etoposide; carboplatin and etoposide; or cyclophosphamide and doxorubicin and vincristine.

The additional anti-cancer therapy may, for example, comprise a combination of a PD-1 axis binding antagonist and cisplatin and 5-FU, or of a PD-1 axis binding antagonist and carboplatin and 5-FU, when the cancer expresses PD-L1 with a combined positivity score of less than 1. The PD-1 axis binding antagonist may, for example, comprise pembrolizumab. The anti-cancer therapy may, for example, comprise a PD-1 axis binding antagonist as monotherapy when the cancer expresses PD-L1 with a combined positivity score (CPS) of ≥1. The PD-1 axis binding antagonist, may for example, comprise pembrolizumab. In either case, the cancer may be recurrent, unresectable or metastatic head and neck cancer. A combined positivity score may be determined by the person skilled in the art, for example by consulting FDA guidelines.

The population of modified T cells and the additional anti-cancer therapy may be administered any number of times, and in any order. The population of modified T cells may, for example, be administered as a single dose. The additional anti-cancer therapy may, for example, be administered (a) before the modified T cells, (b) at the same time as the modified T cells, and/or (c) after the modified T cells. For instance, the additional anti-cancer therapy may, for example, be administered: (a); (b); (c); (a) and (b); (a) and (c); (b) and (c); or (a), (b) and (c). When the additional anti-cancer therapy is administered at the same time as the population of modified T cells, the additional anti-cancer therapy and the population of modified T cells may be comprised in the same composition or in separate compositions.

Administration of the additional anti-cancer therapy at the same time as the modified T cells may refer to administration of the additional anti-cancer therapy and the modified T cells at substantially the same time. In other words, a dose of the additional anti-cancer therapy may be administered at about the same time as a dose of the population of modified T cells. For instance, a dose of the additional anti-cancer therapy may be administered within about 2 hours, about 4 hours, about 6 hours, about 8 hours, about 12 hours, or about 24 hours as a dose of the population of modified T cells.

Administration of the additional anti-cancer therapy before the modified T cells may refer to administration of the anti-cancer therapy at any time before the modified T cells. In other words, a dose of the additional anti-cancer therapy may be administered at any time before a dose of the population of modified T cells. For instance, a dose of the additional anti-cancer therapy may be administered about 24 hours or more, about 36 hours or more, about 48 hours or more, about 72 hours or more, about 4 days or more, about 5 days or more, about 6 days or more, about one week or more, about two weeks or more, about three weeks or more, or about four weeks or more, about five weeks or more, about six weeks or more, about 8 weeks or more, or about 12 weeks or more, before the population of modified T cells. The additional anti-cancer therapy may, for example, be administered before the modified T cells in order to initiate treatment while autologous modified T cells are produced.

Administration of the additional anti-cancer therapy after the modified T cells may refer to administration of the anti-cancer therapy at any time after the modified T cells. In other words, a dose of the additional anti-cancer therapy may be administered at any time after a dose of the population of modified T cells. For instance, a dose of the additional anti-cancer therapy may be administered about 24 hours or more, about 36 hours or more, about 48 hours or more, about 72 hours or more, about 4 days or more, about 5 days or more, about 6 days or more, about one week or more, about two weeks or more, about three weeks or more, or about four weeks or more, about five weeks or more, about six weeks or more, about 8 weeks or more, or about 12 weeks or more, after the population of modified T cells. The additional anti-cancer therapy may, for example, be administered after the modified T cells in order to initiate treatment while autologous modified T cells are produced.

If the additional anti-cancer therapy is administered before the population of modified T cells, the additional anti-cancer therapy may be continued after administration of the modified T cells. Thus, in one aspect of the disclosure, administration of the additional anti-cancer therapy begins before administration of the population of modified T cells, and continues after administration of the population of modified T cells. In other words, a dose of the additional anti-cancer therapy may be administered before a dose of the population of modified T cells, and one or more further doses of the additional anti-cancer therapy may be administered later. The doses of the additional anti-cancer therapy may, for example, be administered in accordance with a known treatment regime for the additional anti-cancer therapy. The purpose of the further doses of the additional anti-cancer therapy may be to maintain the effects achieved by administration of the initial dose. Each of the one or more further doses may comprise the same additional anti-cancer therapy as the initial dose, or a different additional anti-cancer therapy from the initial dose.

If the additional anti-cancer therapy is administered at the same time as the population of modified T cells, the additional anti-cancer therapy may be continued after administration of the modified T cells. Thus, in one aspect of the disclosure, administration of the additional anti-cancer therapy begins at the same time as administration of the population of modified T cells, and continues after administration of the population of modified T cells. In other words, a dose of the additional anti-cancer therapy may be administered at the same time as a dose of the population of modified T cells, and one or more further doses of the additional anti-cancer therapy may be administered later. The doses of the additional anti-cancer therapy may, for example, be administered in accordance with a known treatment regime for the additional anti-cancer therapy. The purpose of the further doses of the additional anti-cancer therapy may be to maintain the effects achieved by administration of the initial dose. Each of the one or more further doses may comprise the same additional anti-cancer therapy as the initial dose, or a different additional anti-cancer therapy from the initial dose.

If the additional anti-cancer therapy is administered after the population of modified T cells, the additional anti-cancer therapy may be continued after initial administration. Thus, in one aspect of the disclosure, administration of the additional anti-cancer therapy begins after administration of the population of modified T cells, and continues after initial administration of the additional anti-cancer therapy. In other words, a dose of the additional anti-cancer therapy may be administered after a dose of the population of modified T cells, and one or more further doses of the additional anti-cancer therapy may be administered later. The doses of the additional anti-cancer therapy may, for example, be administered in accordance with a known treatment regime for the additional anti-cancer therapy. The purpose of the further doses of the additional anti-cancer therapy may be to maintain the effects achieved by administration of the initial dose. Each of the one or more further doses may comprise the same additional anti-cancer therapy as the initial dose, or a different additional anti-cancer therapy from the initial dose.

In any case, the one or more further doses of the additional anti-cancer therapy may be administered at any appropriate interval. Suitable dosage intervals for additional anti-cancer therapy are known in the art and may be peculiar to the identity of the additional anti-cancer therapy.

Any number of further doses of the additional anti-cancer therapy may be administered, such as one or more, two or more, three or more, four or more, five or more, six or more, seven or more, eight or more, nine or more, ten or more, 15 or more, 20 or more, 25 or more, 30 or more, 35 or more, 40 or more, 45 or more or 50 or more further doses. Further doses may be administered until disease progression, unacceptable toxicity, withdrawal of consent, or death.

### Combination Treatment Protocols

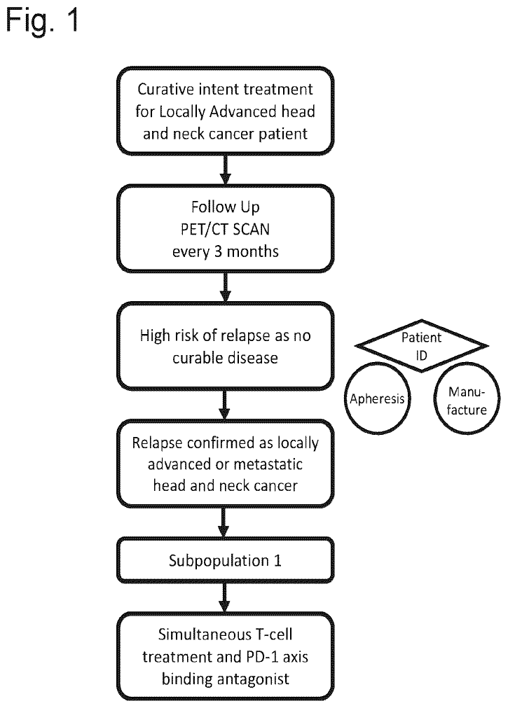

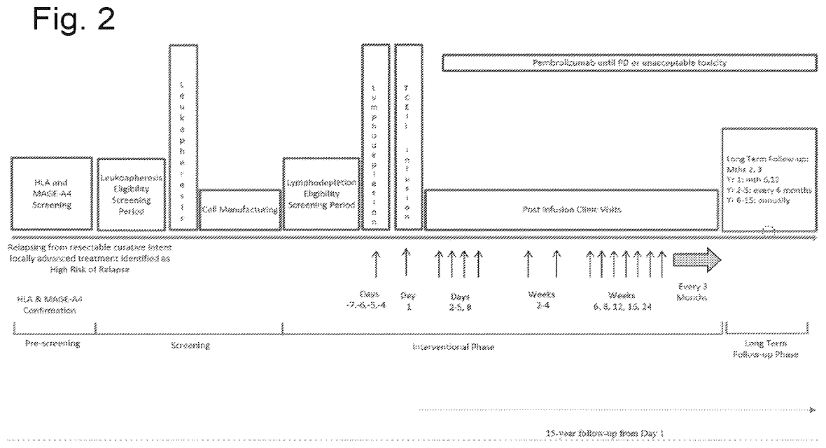

Examples of combination treatment protocols are shown in FIGS. 1 to 4. FIGS. 1 and 2 concern the treatment of head and neck cancer that has relapsed following curative intent treatment for locally advanced head and neck cancer. The treatment is a first-line treatment. In this case, a checkpoint inhibitor (such as a PD-1 axis binding inhibitor, e.g. pembrolizumab) may be administered at the same time, or substantially the same time, as the modified T cells. In other words, a dose of a checkpoint inhibitor may be administered at about the same time as a dose of the population of modified T cells. For instance, a dose of a checkpoint inhibitor may be administered within about 2 hours, about 4 hours, about 6 hours, about 8 hours, about 12 hours, or about 24 hours as a dose of the population of modified T cells. Alternatively, the checkpoint inhibitor may be administered at after the modified T cells. Administration of the checkpoint inhibitor may, for example, be continued after administration of the modified T cells. In other words, a dose of the checkpoint inhibitor may be administered at about the same time as a dose of the population of modified T cells, and one or more further doses of the checkpoint inhibitor may be administered later. The doses of checkpoint inhibitor may, for example, be administered in accordance with a known treatment regime for the checkpoint inhibitor. Treatment regimens for PD-1 axis binding inhibitors (e.g. pembrolizumab) are described above. An additional anti-cancer therapy, such as a chemotherapy, may be administered as an alternative to the checkpoint inhibitor or in addition to the checkpoint inhibitor.

In FIGS. 1 and 2, cells (such as PBMCs) for producing the population of modified T cells are obtained from the individual prior to relapse. For instance, the cells may be obtained when the individual is identified as being at high risk of relapse. This allows production of the population of modified T cells to begin (and, ideally, complete) before the cancer relapses. In turn, this may shorten the window between relapse and administration of the population of modified T cells, i.e. allow the population of modified T cells to be administered as soon as possible after relapsed cancer is diagnosed. This may improve treatment outcomes.

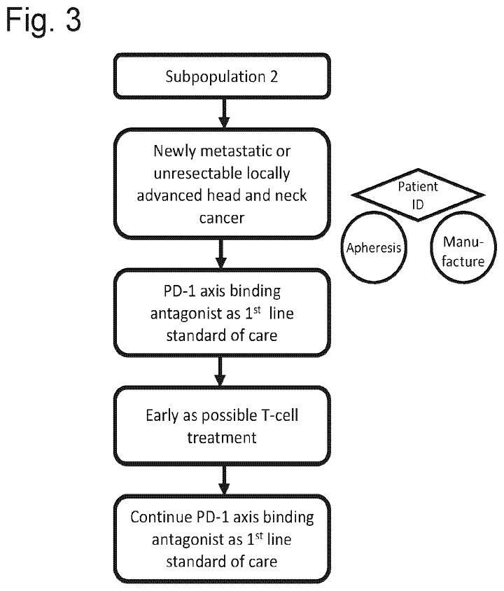

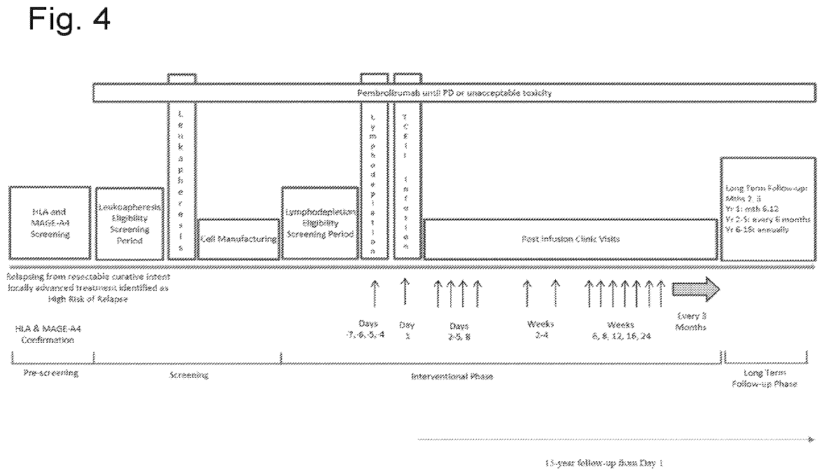

FIGS. 3 and 4 concern the treatment of head and neck cancer that has not previously been treated (such as a newly metastatic or unresectable locally advanced head and neck cancer). The treatment is a first-line treatment. In this case, the checkpoint inhibitor (such as a PD-1 axis binding inhibitor, e.g. pembrolizumab) may be administered before the modified T cells. In other words, a dose of the checkpoint inhibitor may be administered before a dose of the population of modified T cells. For instance, a dose of the checkpoint inhibitor may be administered about 24 hours or more, about 36 hours or more, about 48 hours or more, about 72 hours or more, about 4 days or more, about 5 days or more, about 6 days or more, about one week or more, about two weeks or more, about three weeks or more, or about four weeks or more before the population of modified T cells. Administration of the checkpoint inhibitor may, for example, be continued after administration of the modified T cells. In other words, a dose of the checkpoint inhibitor may be administered before a dose of the population of modified T cells, and one or more further doses of the checkpoint inhibitor may be administered later. The doses of the checkpoint inhibitor may, for example, be administered in accordance with a known treatment regime for the checkpoint inhibitor. Treatment regimens for PD-1 axis binding inhibitors (e.g. Pembrolizumab) are described above. An additional anti-cancer therapy, such as a chemotherapy, may be administered as an alternative to the checkpoint inhibitor or in addition to the checkpoint inhibitor.

In FIGS. 3 and 4, cells (such as PBMCs) for producing the population of modified T cells may be obtained from the individual once the cancer is diagnosed. Production of the population of modified T cells can then begin. Treatment with the checkpoint inhibitor may be implemented during the production window, and the population of modified T cells administered as soon as it is ready. This allows treatment to begin as soon as possible after cancer is diagnosed, which may improve treatment outcomes.

### Administration

As set out above, the population of modified T cells may be administered to the individual as soon as possible after diagnosis of the head and neck cancer. The population may be administered to the individual, for example, less than about 150 days after diagnosis of the head and neck cancer, such as less than about 125 days, less than about 100 days, less than about 90 days, less than about 80 days or less than about 70 days after diagnosis of the head and neck cancer. The population may be administered to the individual, for example, about 30 to about 150 days after diagnosis of the head and neck cancer, such as about 40 to about 125, about 50 to about 100, about 90, about 85, about 80, about 75, about 70, about 65 or about 60 days after diagnosis of the head and neck cancer. Typically, the population of modified T cells is administered as a single dose. One or more (such as two or more, three or more, four or more, or five or more) further doses may though be administered depending on patient factors and the judgement of the practitioner.

The population may comprise any number of modified T cells that will be therapeutically effective. The number of modified T cells for a given individual may depend on factors such as the cancer to be treated, the severity or stage of the cancer, the age of the patient and so on. The number to be administered may thus depend on the judgement of the practitioner and may be peculiar to each subject. By way of example, the population may comprise about 0.8×10⁹ to about 10×10⁹ modified T cells, such as about 0.8×10⁹ to about 1.2×10⁹ modified T cells, about 1.2×10⁹ to about 6×10⁹ modified T cells, or about 1.0×10⁹ to 10×10⁹ modified T cells. The population may, for example, comprise 1.0×10⁹ modified T cells, about 5.0×10⁹ modified T cells, or about 10×10⁹ modified T cells.

Typically, the population of modified T cells is administered intravenously. Any suitable route may though be used, for instance intramuscular, subcutaneous, intradermal, transdermal, or intraperitoneal routes.

The population of modified T cells may be administered in combination with a checkpoint inhibitor and/or an anti-cancer therapy, as set out above. The checkpoint inhibitor or additional anti-cancer therapy may be administered by any route suitable for the given therapy, such as intravenous, intramuscular, subcutaneous, intradermal, transdermal, or intraperitoneal. As set out above, one or more doses of the checkpoint inhibitor or anti-cancer therapy may be administered.

Each dose of the checkpoint inhibitor or anti-cancer therapy may comprise any therapeutically effective amount of the checkpoint inhibitor or anti-cancer therapy. The amount for a given individual may depend on factors such as the cancer to be treated, the severity or stage of the cancer, the age of the patient and so on. The amount to be administered may thus depend on the judgement of the practitioner and may be peculiar to each subject.

By way of example, the checkpoint inhibitor may be a PD-1 axis binding antagonist, such as pembrolizumab. The initial dose of the PD-1 axis binding antagonist, and/or any further dose of the PD-1 axis binding antagonist, may comprise about 200 mg to about 700 mg of the PD-1 axis binding antagonist, such as about 200 mg to about 400 mg of the PD-1 axis binding antagonist. For instance, the initial dose of the PD-1 axis binding antagonist, and/or any further dose of the PD-1 axis binding antagonist, may comprise about 200 mg, about 250 mg, about 300 mg, about 350 mg or about 400 mg of the PD-1 axis binding antagonist. The initial dose of the PD-1 axis binding antagonist, and/or any further dose of the PD-1 axis binding antagonist, may, for example, comprise about 200 mg to about 400 mg of the PD-1 axis binding antagonist, such as about 480 mg of the PD-1 axis binding antagonist. In a preferred aspect of the disclosure, the initial dose of the PD-1 axis binding antagonist, and any further dose of the PD-1 axis binding antagonist, comprises 200 mg Q3W of pembrolizumab or 400 mg Q6W of pembrolizumab.

In some aspects of the disclosure, the method comprises administering lymphodepleting chemotherapy to the individual prior to administration of the population of modified T cells. That is, lymphodepleting chemotherapy may be administered before step (a). Lymphodepleting chemotherapy may, for example, be administered from about 14 days before step (a) to about 1 day before step (a), such as about 13 days before step (a) to about 2 days before step (a), about 12 days before step (a) to about 3 days before step (a), about 11 days before step (a) to about 4 days before step (a), about 10 days before step (a) to about 5 days before step (a), about 9 days before step (a) to about 6 days before step (a), about 8 days before step (a) to about 7 days before step (a), about 10 days before step (a) to about 1 day before step (a), about 9 days before step (a) to about 2 days before step (a), about 8 days before step (a) to about 3 days before step (a), about 7 days before step (a) to about 4 days before step (a), or about 6 days before step (a) to about 5 days before step (a). Preferably, lymphodepleting chemotherapy is administered from about 7 days before step (a) to about 4 days before step (a). The purpose of lymphodepleting chemotherapy may be to deplete the lymphocyte compartment of the individual, so as to provide space into which the adoptively-transferred modified T cells can expand. In this way, the effects of a given dose of the modified T cells can be maximised. The lymphodepleting chemotherapy may comprise any suitable lymphotoxic agent. Lymphotoxic agents and suitable dosages are known in the art. The lymphodepleting chemotherapy may, for example, comprise fludarabine and/or cyclophosphamide. Typically, the lymphodepleting chemotherapy is administered intravenously. Any suitable route may though be used, for instance intramuscular, subcutaneous, intradermal, transdermal, or intraperitoneal routes.

### Medicaments and Medical Uses

The disclosure provides a population of modified T cells comprising a heterologous CD8 co-receptor and a heterologous T-cell receptor capable of binding to a peptide antigen of MAGE-A4 for use in a method of treating head and neck cancer in an individual. Any of the aspects described above in connection with the method of the disclosure may also apply to the population for use.

The disclosure also provides the use of a population of modified T cells in the manufacture of a medicament for use in a method of treating head and neck cancer in an individual, wherein the modified T cells comprise a heterologous CD8 co-receptor and a heterologous T-cell receptor capable of binding to a peptide antigen of MAGE-A4. Any of the aspects described above in connection with the method of the disclosure may also apply to this use of the population.

The disclosure also provides the use of a population of modified T cells in a method of treating head and neck cancer in an individual, wherein the modified T cells comprise a heterologous CD8 co-receptor and a heterologous T-cell receptor capable of binding to a peptide antigen of MAGE-A4. Any of the aspects described above in connection with the method of the disclosure may also apply to this use of the population.

### Examples

### Introduction

ADP-A2M4CD8 specific peptide enhanced affinity receptor (SPEAR™) T cells are genetically engineered to target the tumour antigen MAGE-A4 in the context of the appropriate human leukocyte antigen (HLA) expression. ADP-A2M4CD8 are autologous CD4 and CD8 positive T cells that have been transduced with a self-inactivating (SIN) lentiviral vector expressing a high affinity MAGE-A4 specific T cell receptor (TCR) and an additional CD8α co-receptor.

The affinity-optimised TCR (ADP-A2M4 TCR) comprises an alpha chain variable domain comprised in SEQ ID NO: 2, and a beta chain variable domain comprised in SEQ ID NO: 3. When expressed in a T cell, the signal peptides are cleaved from SEQ ID NOs: 2 and 3 prior to surface expression. A2M4 TCR targets the tumour antigen MAGE-A4 and activates engineered T cells. It recognizes the MAGE-A4230-239 (GVYDGREHTV; SEQ ID NO: 1) peptide sequence derived from MAGE-A4, when presented in the HLA-A*02-GVYDGREHTV antigen complex.

The CD8α co-receptor comprised in ADP-A2M4CD8 SPEAR™ T cells is designed to provide additional functionality to CD4 T cells. Because CD4+ T cells have a weak effector function in response to Class I antigens, a CD8α co-receptor was introduced alongside the TCR, in order to increase TCR binding avidity and enhance the polyfunctional response of engineered CD4+ T cells against MAGE-A4 positive tumour. The co-expression of CD8α adds CD8+ killer T cell capability to CD4+ helper T cells, while also maintaining/enhancing the helper cell capabilities of CD4+ T cells. The addition of a CD8α co-receptor directly impacts TCR binding to the HLA-peptide complex in CD4+ T cells, enhancing CD4+ T cell effector function. ADP-A2M4CD8 SPEAR™ T cells are therefore designed to improve upon ADP-A2M4 expressing T cells.

This has been confirmed in preclinical in vitro assays, in which ADP-A2M4CD8 showed a clear improvement in T cell activation (when cultured with antigen positive cells) relative to ADP-A2M4 expressing T cells, as measured by increased CD40L surface expression, particularly in the CD4+ fraction. When dendritic cells (DCs) were included in co-cultures, a marked improvement was seen with ADP-A2M4CD8 T cells. Cytokine release from both DCs (IL-12, MIG) and T cells (IFNy, IL-2 and other Th1) was improved compared to cultures containing the ADP-A2M4 cells. Additionally, a conversion of CD4+ T cells was seen, from being unable to kill MAGE-A4 positive 3D microspheres, to having an effective cytotoxic function when transduced with ADP-A2M4CD8. Therefore, CD4+ T cells transduced with ADP-A2M4CD8 display not only CD4+ helper functions, but also improved T cell effector functions.

Individuals with recurrent or metastatic head and neck cancer have response rates around 20% for checkpoint inhibitor monotherapy and 36% for checkpoint inhibitors in combination with chemotherapy, with progression-free survival of less than 5 months. Preliminary clinical trials results in connection with ADP-A2M4CD8 demonstrate that a response rate of approximately 36% is observed when ADP-A2M4CD8 is used for the treatment of cancer including head and neck cancers when previous treatment using the standard of care has failed. Within this, there were 3 confirmed responses out of 4 individuals with late-stage, metastatic head and neck cancer who received ADP A2M4CD8 monotherapy.

An improved regimen for treatment of head and neck cancer is desired. Examples 1 and 2 consider regimens in which ADP-A2M4CD8 is used as a first line treatment for (1) relapsed head and neck cancer and (2) newly metastatic or unresectable locally advanced head and neck cancer respectively.

### Example 1. Relapsed Head and Neck Cancer

Subjects are selected for treatment with ADP-A2M4CD8. In brief, subjects eligible for selection must have been previously diagnosed with locally advanced head and neck cancer and subjected to curative intent treatment, such as surgical resection and/or radiotherapy. Subjects eligible for selection typically receive a follow up PET and/or CT scan every three months following the curative intent treatment. The scans identify whether or not a subject is at high risk of relapse. Subjects at high risk of relapse may be selected for treatment.

In addition, selected subjects are positive for HLA-A*02:01, HLA-A*02:03, or HLA-A*02:06, or another HLA-A*02 allele having the same protein sequence in the peptide binding domains. Patients who are HLA-A*02:05 positive are excluded from the study, because alloreactivity from ADP-A2M4 and from ADP-A2M4CD8 has previously been seen towards two HLA-A*02:05 positive cell lines. Patients with either HLA-A*02:07 or any A*02 null allele as the sole HLA-A*02 allele are also excluded due to decreased activity with these alleles.

Selected subjects also have a tumour that shows MAGE-A4 expression defined as ≥30% of tumour cells that are ≥2+ by immunohistochemistry (IHC).

Autologous cells are collected from selected subjects by leukapheresis for processing and manufacture into ADP-A2M4CD8. The heterologous TCR comprised in ADP-A2M4CD8 T cells comprises an alpha chain sequence comprised in SEQ ID NO: 2 and a beta chain sequence comprised in SEQ ID NO: 3. The heterologous CD8 co-receptor comprised in ADP-A2M4CD8 T cells comprises two CD8 alpha chains, each comprising an amino acid sequence of SEQ ID NO: 10. The surface-expressed heterologous TCR and surface-expressed heterologous CD8 co-receptor do not comprise the signal sequence. The autologous cells are preferably collected prior to diagnosis of relapsed locally advanced or metastatic head and neck cancer.

A baseline tumour assessment is obtained prior to treatment. Then, subjects are provided with lymphodepleting chemotherapy using fludarabine and cyclophosphamide (Day −7 through Day −4) in anticipation of administration of ADP-A2M4CD8. Subjects receive a single intravenous infusion of ADP-A2M4CD8 on Day 1. Treatment with a standard-of-care anti-cancer drug therapy, typically pembroluzimab, is implemented at the same time (e.g. within around 14 days of ADP-A2M4CD8 administration).

Between 1×10⁸ to 1×10¹⁰ ADP-A2M4CD8 T cells are administered to the subjects. The initial dose selected for ADP-A2M4CD8 is 1×10⁹ transduced cells (Range: 0.8×10⁹-1.2×10⁹ transduced cells).

Subjects are monitored immediately post-infusion monitoring (Day 1 through Day 8). Subjects are monitored weekly until Week 4 post-infusion. Then, subjects are monitored at Weeks 6, 8, 12, 16, and 24 and at least every 3 months thereafter until disease progression. Tumour response is assessed according to response evaluation criteria in solid tumours (RECIST) v1.1. Additional data collected includes:
Core needle biopsies, to directly evaluate the “immune landscape” inside the tumour at baseline and during the course of the study.
Cytokine levels in the serum at baseline and during the course of the study.
Humoral immune responses to tumour antigens at baseline and during the course of the study, using serum.
Antibodies to ADP-A2M4CD8 at baseline and during the course of the study, using serum.
Soluble markers representing the tumour and its microenvironment, using liquid biopsies. For instance, markers of circulating tumour cells (CTCs), exosome, and cell-free DNA (cfDNA) produced by dying tumour cells) may be used to monitor both the molecular signature of the tumour burden (including the expression of the target antigen) and the immune response. The analysis of such soluble markers allows estimation and genetic profiling of the global tumour burden, including expression of MAGE-A4 mRNA and mutational profiling. The analysis of such soluble markers also allows systemic assessment of the immune response.
The phenotype and activity of the gene-modified T cells before and after infusion. The relevant assays may be performed using blood and, if resection is performed, tumour. The assays include: (i) phenotype analysis for determination of T-cell lineages in cell product and in the blood (and, if resection performed, tumour) post-infusion; (ii) quantitation of the senescence and activation status of immune subsets from PBMCs; (iii) analysis of gene expression or epigenetic profile to reflect phenotype and functional state of the cells; and/or (iv) direct functional assessment of the cells.
Persistence of infused engineered cells, and correlation with therapeutic effect. Persistence may be determined by the copies of gene-modified DNA per g DNA, and/or data on the number of transduced cells per L or relative to total lymphocyte number. Well-established methodologies include (i) quantitation of ADP-A2M4CD8 cells by quantitative PCR of transgene from DNA extracted from frozen PBMCs, and (ii) quantitation and phenotyping of ADP-A2M4CD8 cells by flow cytometry, DNA and RNA analysis from frozen PBMCs.

FIGS. 1 and 2 summarise the process described above.

Doses of up to 10×10⁹ ADP-A2M4CD8 have been administered and shown to be well-tolerated. Emerging data indicates that treatment outcomes may be improved when first-line treatment of relapsed head and neck cancer comprises administration of ADP-A2M4CD8. Outcomes may, for instance, be improved relative to a “standard-of care” first-line treatment of relapsed head and neck cancer, such as checkpoint inhibitor (e.g. PD-1 axis binding antagonist) monotherapy or checkpoint inhibitors in combination with chemotherapy.

### Example 2. Newly Metastatic or Unresectable Locally Advanced Head and Neck Cancer

Subjects are selected for treatment with ADP-A2M4CD8. In brief, selected subjects have newly metastatic or unresectable locally advanced head and neck cancer. Selected subjects are also positive for HLA-A*02:01, HLA-A*02:03, or HLA-A*02:06, or another HLA-A*02 allele having the same protein sequence in the peptide binding domains. Patients who are HLA-A*02:05 positive are excluded from the study, because alloreactivity from ADP-A2M4 and from ADP-A2M4CD8 has previously been seen towards two HLA-A*02:05 positive cell lines. Patients with either HLA-A*02:07 or any A*02 null allele as the sole HLA-A*02 allele are also excluded due to decreased activity with these alleles. In addition, selected subjects have a tumour that shows MAGE-A4 expression defined as ≥30% of tumour cells that are ≥2+ by immunohistochemistry (IHC).

Autologous cells are collected from enrolled subjects by leukapheresis for processing and manufacture into ADP-A2M4CD8. The heterologous TCR comprised in ADP-A2M4CD8 T cells comprises an alpha chain sequence comprised in SEQ ID NO: 2 and a beta chain sequence comprised in SEQ ID NO: 3. The heterologous CD8 co-receptor comprised in ADP-A2M4CD8 T cells comprises two CD8 alpha chains, each comprising an amino acid sequence of SEQ ID NO: 10. The surface-expressed heterologous TCR and surface-expressed heterologous CD8 co-receptor do not comprise the signal sequence.

A baseline tumour assessment was obtained prior to treatment. Then, the subjects are administered one or more cycles of a standard-of-care anti-cancer drug therapy, typically pembroluzimab. Subjects are provided with lymphodepleting chemotherapy using fludarabine and cyclophosphamide (Day −7 through Day −4) in anticipation of administration of ADP-A2M4CD8. Subjects receive a single intravenous infusion of ADP-A2M4CD8 on Day 1. Day 1 is set to be as soon as possible after diagnosis of newly metastatic or unresectable locally advanced head and neck cancer, i.e. as soon as autologous cells are ready. This is typically around 60 days from leukapheresis.

Between 1×10⁸ to 1×10¹⁰ ADP-A2M4CD8 T cells are administered to the subjects. The initial dose selected for ADP-A2M4CD8 is 1×10⁹ transduced cells (Range: 0.8×10⁹-1.2×10⁹ transduced cells).

Subjects are monitored immediately post-infusion monitoring (Day 1 through Day 8). Subjects were monitored weekly until Week 4 post-infusion. Then, subjects are monitored at Weeks 6, 8, 12, 16, and 24 and at least every 3 months thereafter until disease progression. Tumour response is assessed according to response evaluation criteria in solid tumours (RECIST) v1.1. Additional data collected includes:
Core needle biopsies, to directly evaluate the “immune landscape” inside the tumour at baseline and during the course of the study.
Cytokine levels in the serum at baseline and during the course of the study.
Humoral immune responses to tumour antigens at baseline and during the course of the study, using serum.
Antibodies to ADP-A2M4CD8 at baseline and during the course of the study, using serum.
Soluble markers representing the tumour and its microenvironment, using liquid biopsies. For instance, markers of circulating tumour cells (CTCs), exosome, and cell-free DNA (cfDNA) produced by dying tumour cells) may be used to monitor both the molecular signature of the tumour burden (including the expression of the target antigen) and the immune response. The analysis of such soluble markers allows estimation and genetic profiling of the global tumour burden, including expression of MAGE-A4 mRNA and mutational profiling. The analysis of such soluble markers also allows systemic assessment of the immune response.
The phenotype and activity of the gene-modified T cells before and after infusion. The relevant assays may be performed using blood and, if resection is performed, tumour. The assays include: (i) phenotype analysis for determination of T-cell lineages in cell product and in the blood (and, if resection performed, tumour) post-infusion; (ii) quantitation of the senescence and activation status of immune subsets from PBMCs; (iii) analysis of gene expression or epigenetic profile to reflect phenotype and functional state of the cells; and/or (iv) direct functional assessment of the cells.
Persistence of infused engineered cells, and correlation with therapeutic effect. Persistence may be determined by the copies of gene-modified DNA per g DNA, and/or data on the number of transduced cells per L or relative to total lymphocyte number. Well-established methodologies include (i) quantitation of ADP-A2M4CD8 cells by quantitative PCR of transgene from DNA extracted from frozen PBMCs, and (ii) quantitation and phenotyping of ADP-A2M4CD8 cells by flow cytometry, DNA and RNA analysis from frozen PBMCs.

FIGS. 3 and 4 summarise the process described above.

Doses of up to 10×10⁹ ADP-A2M4CD8 have been administered and shown to be well-tolerated. Emerging data indicates that treatment outcomes may be improved when first-line treatment of newly metastatic or unresectable locally advanced head and neck cancer comprises administration of ADP-A2M4CD8. Outcomes may, for instance, be improved relative to a “standard-of care” first-line treatment of newly metastatic or unresectable locally advanced head and neck cancer, such as checkpoint inhibitor (e.g. PD-1 axis binding antagonist) monotherapy or checkpoint inhibitors in combination with chemotherapy.

### Example 3. Preliminary Results of the Phase 1 SURPASS Trial of ADP-A2M4CD8, a Next-Generation SPEAR T-Cell Therapy, in Patients with Head and Neck Cancer

Autologous T-cells were obtained by leukapheresis, transduced with a self-inactivating lentiviral vector expressing the MAGE-A4-specific TCR and the CD8α co-receptor, and infused back to the patients as ADP-A2M4CD8 following lymphodepleting chemotherapy.

Four patients with MAGE-A4-positive head and neck cancer were treated with ADP-A2M4CD8. Prior therapies were:
One line: 5-fluorouracil (5fu), carboplatin, pembrolizumab;
Three lines: cisplatin, carboplatin, paclitaxel, pembrolizumab, cetuximab, panitumumab;
Three lines: 5fu, carboplatin, cetuximab, nivolumab, taxane, cetuximab;
Five lines: cisplatin, 5fu, paclitaxel, cetuximab, carboplatin, nivolumab, CX-2029.

Three of the four head and neck cancer patients were screened for human papillomavirus; all were negative. The baseline characteristics of the patients are provided in the table below:

| Characteristic | Head and neck cancer (n = 4) |
|---|---|
| Sex, n (%) | |
| Male | 2 (50.0) |
| Female | 2 (50.0) |
| Age, years, median (range) | 59.5 (43-70) |
| H-score^{a}, median (range) | 287.5 (140-300) |
| ECOG at baseline, n (%) | |
| 0 | 1 (25) |
| 1 | 3 (75) |
| Systemic bridging therapy, | 2 (50) |
| yes, n (%) | |
| Transduced cells, ×10⁹ cells, | 5.46 (4.6-6.5) |
| median (range) | |
| Prior lines of systemic therapy, | 3 (1-5) |
| median (range)) | |
| Baseline SLD, median (range) | 69 (19-164) |

^{a}H-score: 1 × (% of 1+ cells) + 2 × (% of 2+ cells) + 3 × (% of 3+ cells). ECOG, Eastern Cooperative Oncology Group;
SLD, sum of the longest diameters of target lesions/

Of the patients who received systemic bridging therapy, one patient received paclitaxel and cetuximab, and one patient received tipifarnib.

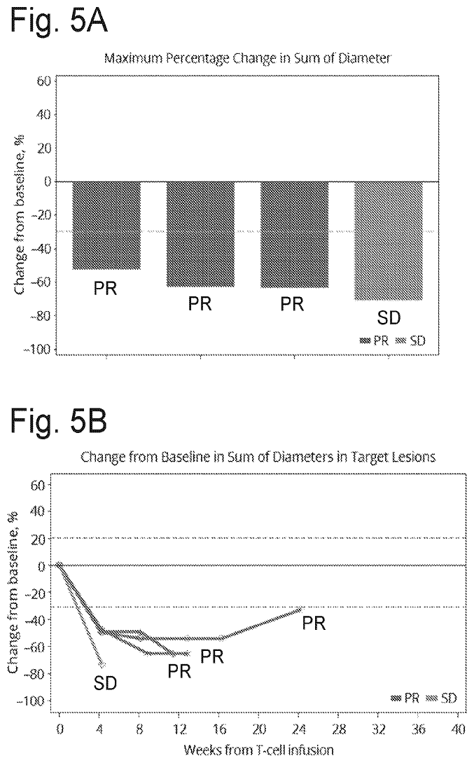

The overall response rate (ORR) per Response Evaluation Criteria in Solid Tumours (RECIST) v1.1 by investigator review was 75.0% (3 partial responses; FIG. 5).

The disease control rate was 100.00% (3 partial responses and one stable disease).

The median duration of the response was 8.7 weeks, with a range of 7.4 to 20.1 weeks. The data shown in FIG. 5 indicates changes from baseline SLD through progression of disease or prior to surgical resection.

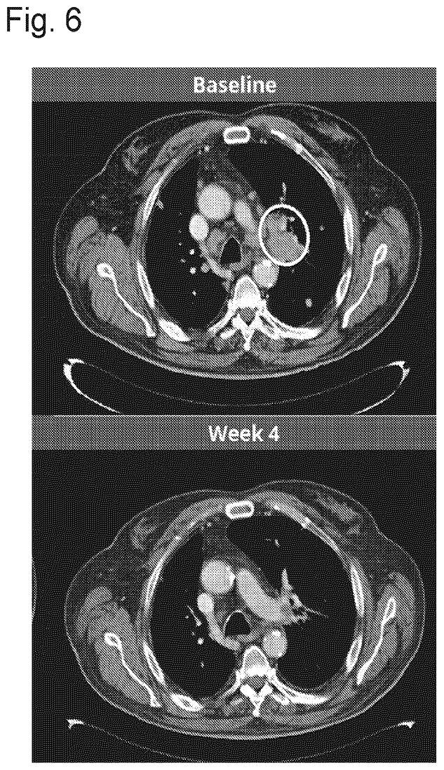

FIG. 6 looks at the hilar mass of a patient with stage IV head and neck cancer who had a confirmed partial response following therapy with ADP-A2M4CD8. The patient was a 69-year old white man with stage IV squamous head and neck cancer. The MAGE-A4 expression in the tumour cells was: 85% 3+, 10% 2+ and 5% 1+. The baseline SLD was 111 mm (from 5 target lesions). Prior systemic therapies were platinum-based therapy, nivolumab, and taxane/cetuximab. The patient was treated with 5 billion transduced ADP-A2M4CD8 T cells. A confirmed response was initially reported at week 4 and was durable to week 24.

1. A method of treating head and neck cancer in an individual, comprising administering to the individual a population of modified T cells comprising a heterologous CD8 co-receptor and a heterologous T-cell receptor (TCR) capable of binding to a peptide antigen of MAGE-A4.

2. The method of claim 1, wherein the method further comprises administering a checkpoint inhibitor to the individual, optionally wherein the population of modified T cells and the checkpoint inhibitor are administered in the same line of therapy.

3. The method of claim 2, wherein:
(a) administration of the checkpoint inhibitor begins before administration of the population of modified T cells, and continues after administration of the population of modified T cells; or
(b) administration of the checkpoint inhibitor begins at the same time as or after administration of the population of modified T cells, and continues after administration of the population of modified T cells.

4. The method of claim 2, the checkpoint inhibitor comprises a Programmed Death-1 (PD-1) axis binding antagonist, optionally pembrolizumab.

5. The method of claim 1, wherein the method further comprises administering an additional anti-cancer therapy to the individual, optionally wherein:
(a) population of modified T cells and the additional anti-cancer therapy are administered in the same line of therapy; and/or
(b) the additional anti-cancer therapy is a chemotherapy.

6. The method of claim 5, wherein:
(a) administration of the additional anti-cancer therapy begins before administration of the population of modified T cells, and continues after administration of the population of modified T cells; or
(b) administration of the additional anti-cancer therapy begins at the same time as or after administration of the population of modified T cells, and continues after administration of the population of modified T cells.

7. The method of claim 1, wherein the head and neck cancer is relapsed head and neck cancer.

8. The method of claim 7, wherein the head and neck cancer has relapsed following curative intent treatment for locally advanced head and neck cancer, optionally wherein:
(a) the curative intent treatment comprises surgical resection and/or radiation therapy; and/or
(b) the curative intent treatment comprises systemic therapy.

9. The method of claim 7, wherein:
(a) the method comprises administering a checkpoint inhibitor to the individual and administration of the checkpoint inhibitor begins at the same time as or after administration of the population of modified T cells and continues after administration of the population of modified T cells; and/or
(b) the method comprises administering an additional anti-cancer therapy to the individual and administration of the additional anti-cancer therapy begins at the same time as or after administration of the population of modified T cells and continues after administration of the population of modified T cells, optionally wherein the additional anti-cancer therapy is a chemotherapy.

10. The method of claim 1, wherein the individual has not previously been treated for the head and neck cancer, optionally wherein the head and neck cancer is newly metastatic or unresectable locally advanced head and neck cancer.

11. The method of claim 10, wherein:
(a) the method comprises administering a checkpoint inhibitor to the individual, and administration of the checkpoint inhibitor begins before or at the same time as administration of the population of modified T cells and continues after administration of the population of modified T cells; or
(b) the method comprises administering an additional anti-cancer therapy to the individual, and administration of the additional anti-cancer therapy begins before or at the same time as administration of the population of modified T cells and continues after administration of the population of modified T cells, optionally wherein the additional anti-cancer therapy is a chemotherapy.

12. The method of claim 1, wherein:
(a) the population of modified T cells is administered as soon as possible after diagnosis of the head and neck cancer; and/or
(b) the population of modified T cells is administered as a single dose.

13. The method of claim 1, wherein:
(a) the heterologous TCR binds to GVYDGREHTV (SEQ ID NO: 1) in complex with an HLA molecule;
(b) the heterologous TCR comprises an alpha chain amino acid sequence having at least 80% sequence identity to SEQ ID NO: 2 and a beta chain amino acid sequence having at least 80% sequence identity to SEQ ID NO: 3; and/or
(c) the CD8 co-receptor is CD8α.

14. The method of claim 1, wherein the modified T cells are autologous with respect to the individual, optionally wherein the method comprises producing the population by:
(a) obtaining peripheral blood mononuclear cells (PBMCs) from the individual;
(b) selecting T cells from the PBMCs; and
(c) modifying the selected T cells to express the heterologous CD8 co-receptor and the heterologous TCR;
optionally wherein the head and neck cancer is relapsed head and neck cancer and wherein at least one of step (a) to (c) are performed prior to relapse.

15. A population of modified T cells comprising a heterologous CD8 co-receptor and a heterologous T-cell receptor capable of binding to a peptide antigen of MAGE-A4, for use in the method of claim 1.


SEQUENCE LISTING (32 sequences)

SEQ ID NO: 1 - protein, 10 aa, Homo sapiens
GVYDGREHTV

SEQ ID NO: 2 - protein, 282 aa, synthetic construct
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKR

SEQ ID NO: 3 - protein, 311 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 4 - protein, 6 aa, synthetic construct
VSPFSN

SEQ ID NO: 5 - protein, 6 aa, synthetic construct
LTFSEN

SEQ ID NO: 6 - protein, 15 aa, synthetic construct
CVVSGGTDSWGKLQF

SEQ ID NO: 7 - protein, 5 aa, synthetic construct
KGHDR

SEQ ID NO: 8 - protein, 6 aa, synthetic construct
SFDVKD

SEQ ID NO: 9 - protein, 14 aa, synthetic construct
CATSGQGAYEEQFF

SEQ ID NO: 10 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 11 - protein, 9 aa, Homo sapiens
VLLSNPTSG

SEQ ID NO: 12 - protein, 8 aa, Homo sapiens
YLSQNKPK

SEQ ID NO: 13 - protein, 6 aa, Homo sapiens
LSNSIM

SEQ ID NO: 14 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 15 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 16 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 17 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 214 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 29 - protein, 229 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 30 - protein, 213 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC